SEQUENCE LISTING (9 sequences)

SEQ ID NO: 1 - DNA, 4123 bp, Artificial Sequence
  Description of Artificial Sequence; Note = synthetic construct
gaattccactctgaaagtgggagaggatccaagtagggaaggagaaagggtacaaaatgacctgtcccaagaaatggactggatctttcaatcatttactcatccaacaaatatttgaagttgtaaaatgaccacaaagtgggctaaaagttcagacgtatggagcatgtccctctcggtctttggttttgaccaaagctcagaattgtggaaagaaagaaaaagtagtgggttatgcatgttgtgtcacagtggaagatgaagtagtgggtgttaaagaaaatgtttggatagataaaggatcaagtgagcggcaaacacacattcctggcagagtgaatgggctggctttctagagattcttgttaaaataccttttgtgtttgcctctttgtggtcttcacaactaggattaatttagggaagataatcatgatccaggtgaggataaagattccagagaaaggcttatttctaccccttaacttctttgtttttcttcctttctaaaagttttgtcatttttaaaatttattttttatttaatttttttcatgcaatataatttgatcatattctttccttcctccaacttctcctagatcctcagggccttcctagctatccatcttcatgttaatggatagactgacaaccaaaacattctttctctgcttaaataatatctccataaaatctataaataaatgaggtagttggaaactatctcagcacttttcaattgattggctagtaatccttcaatatctcattttttttaactttcgctttatctattctgtgtgnacattaatttttttcaggcaaggcataatatatatataattggactgatttctttattagagtttgccctatgtgaggtcaagaaatattcttaaattaatgagtgactgaataagtgatgggcaatttaagtatttagaaaagaaaggttttattattccattcagtcaagatagtgagacagagaaagagtctgtcacaggctgtgtatgtggtgaggctgattgagtcttgagccacctgaatgcaactgcactgttccacctgctggcacatccatcctggatcaatctggagtgtgactgtgacaagtctcagataaaatggaagaaacagctggatttggagtccagatgcaaagatgactataggtagaaactttcagcaattacattcatctgaacacaccaactactgttgtcatcatttcaccctgaaattaggaaaatagtacaagcagctacacctattacatgtttggtaaattagaatgtgaatttcttaatatccaggttaatgtctagtccatgactttacctcatcagcaaggatatacataacatgcaatatgtgctcaataaatagttgtgagtagttcagagaaatgggaattggtatacatatagatgttaccaagactagatactagagatttgtttttactgtttaccaaagctgatgttgcagattaataaactttggattctgaggtcagtctctgtctgtcttctccattccccctcccacaagtaggtgtgtctaccttctcatgacttaaatgcgggtttctaaacatttagtgacactagtgatccagaaactactaaccatgggtttttttttatttagccctacaaggtacttggatggtatctctgggttcttccaatgggccttcttgctcagtctatttctggtgctgttcaaggcagtccaattctacttacgaaggcagtggctgctcaagaccctccagcatttcccatgcatgccttcccactggctttgggggcaccatctgaagggacaaggagctccagcagattcttatatgggtagagaaattcccaagtgcctgcttacagtgtctctcggggagcaatatacgagtcctgctttatgatcctgactatgtgaaggtggttctggggagatcagatccaaaggcttctggaatttatcaattctttgctccctggattggttatggtttgctcctgttgaatgggaagaagtggttccagcatcggcggatgttgactccagccttccactatgacatcctcaaaccctatgtcaaaatcatggcggactctgtcaatataatgctagataaatgggagaagcttgatggccaggaccaccctctggagatcttccactgtgtttcattgatgacactggacactgttatgaagtgtgctttcagctaccaaggcagtgttcagttggatgaaaattccaagttgtatactaaggctgtcgaggatctaaacaacctgactttctttcgcctgcggaatgccttttataagtacaacatcatctacaatatgtcctctgatggacgtttgtcccaccatgcctgccagattgctcacgagcacacagatggagtgatcaagatgaggaagtctcagctgcagaatgaggaagagctgcagaaggccaggaagaagagacacttggatttcttggacatcctcttgtttgccagaatggaggataggaacagcttgtctgatgaggacctgcgtgcagaggtggacacattcatgtttgagggtcatgacactacagccagtggaatttcctggattttctatgctctggccacccaccctgagcaccaacagagatgcagagaggaggtgcagagcattctgggtgatggaacctctgtcacatgggaccatctgggccagatgccctacaccaccatgtgcatcaaggaggccctgaggctctatccaccagtaatatctgtgagtcgagagctcagctcacctgtcaccttcccagatggacgctccatacccaaaggtatcacagccacaatttccatttatggcctacatcataacccacgtttctggccaaacccaaaggtgtttgacccctctagatttgcaccagattcttctcaccatagccatgcttatctgccattctcaggaggatcaaggaactgcattgggaaacagtttgctatgaacgagctgaaggtggctgtggccctgaccctgcttcgctttgaattgctgccagatcccaccaggatcccagtccccattgcaagacttgtgttgaagtccaagaatgggatccacctgtgtctcaagaagctaagataattctgatggagtcagggcagctccagaggtctgctgcctgcaatacctgcttttgtctctggcttttctgtactttgctttctctttgattcccattcttctgctctctgcaatgtgtcctgtcatctcatctttctgccctcatttctgtagcttttcctctagacaccttcctaacctgtgcatgtacctgtttcccatctcgctttaactctgaccagccactgaacctgcagccagcagcctgtcccccagcctgttcacccctcataaccattgcactgacagaggaagatatattttagagggagacacttgtacctttctctcccttcagttattagactcttgggacaatggacatcatgaattaaaacgttcttagaaatcacatgctgggagaaaattaacactaaaatctggtaccagccagaggaaggaacttgactcaaaataagagatttttagatatttctgtctgtctcatagttaaaattaatgttttcctgctttctggcatatgcctcatcttttctatgaagtagtaatactgatacagaaaggtagagagaaatgaatagtttttgctactttgggccaaactgtgaaaaaatccattttatttcatcaatttctgtttcccaatttcatttaagacacaggaaaactactcagcatgaactttggggagccagagcagttttggcaatccagggaagcatgttgccatctggtccctactgttagaatgtggtagaattctcagctcctgagaggttgttctctgcttttgactcctgagctggttgtgtagaaatgcaggttggcgttttttgtgaagctaaggagttttctgactttaacccggtcttatttgtttagagtactctgattattcactttagtgatttggagaattcctattaaaatcacatgacatggaaaaaaaaaaaaaaaggaat

SEQ ID NO: 2 - protein, 507 aa, Artificial Sequence
  Description of Artificial Sequence; Note = synthetic construct
MGFFVFSPTRYLDGISGFFQWAFLLSLFLVLFKAVQFYLRRQWLLKTLQHFPCMPSHWLWGHHLKDKELQQILIWVEKFPSACLQCLSGSNIRVLLYDPDYVKVVLGRSDPKASGIYQFFAPWIGYGLLLLNGKKWFQHRRMLTPAFHYDILKPYVKIMADSVNIMLDKWEKLDGQDHPLEIFHCVSLMTLDTVMKCAFSYQGSVQLDENSKLYTKAVEDLNNLTFFRLRNAFYKYNIIYNMSSDGRLSHHACQIAHEHTDGVIKMRKSQLQNEEELQKARKKRHLDFLDILLFARMEDRNSLSDEDLRAEVDTFMFEGHDTTASGISWIFYALATHPEHQQRCREEVQSILGDGTSVTWDHLGQMPYTTMCIKEALRLYPPVISVSRELSSPVTFPDGRSIPKGITATISIYGLHHNPRFWPNPKVFDPSRFAPDSSHHSHAYLPFSGGSRNCIGKQFAMNELKVAVALTLLRFELLPDPTRIPVPIARLVLKSKNGIHLCLKKLR

SEQ ID NO: 3 - protein, 508 aa, Artificial Sequence
  Description of Artificial Sequence; Note = synthetic construct
MSASALSSIRFPGSISEYLQVASVLSLLLLLFKTAQLYLHRQWLLSSTQQFPSPPSHWLFGHKILKDQDLQDILTRIKNFPSACPQWLWGSKVRIQVYDPDYMKLILGRSDPKANGSYRFLAPWIGRGLLMLDGQTWFQHRRMLTPAFHYDILKPYTEIMADSVRVMLDKWEQIVGQDSTLEIFRHITLMTLDTIMKCAFSHEGSVQLDRKYKSYIQAVEDLNDLVFSRVRNIFHLNDIIYRVSSNGCKANSACQLAHDHTDQVIKSRRIQLQDEEELEKLKKKRRLDFLDILLFARMENGKSLSDKDLRAEVDTFMFEGHDTTASGISWIFYALATNPEHQQRCRKEIQSLLGDGTSITWNDLDKMPYTTMCIKEALRIYPPVPSVSRELSSPVTFPDGRSLPKGIHVMLSFYGLHHNPTVWPNPEVFDPSRFAPGSSRHSHSFLPFSGGARNCIGKQFAMNELKVAVALTLLRFELLPDPTRVPIPIPRIVLKSKNGIHLHLKELQ

SEQ ID NO: 4 - DNA, 2116 bp, Artificial Sequence
  Description of Artificial Sequence; Note = synthetic construct
tgaattcatcttctaccagctctttctgtaaatatttcaaatatttacacaaatatttgtgaactgtttggataaagtgacaccactattacctaatatgtctttcatttcattgctccccaaagaggctgttcaggtccatcaaccctggtcttgaaatcaagctctgctcacacccctctccctcccccaagtaggtggggcaaccctcctggggtttgcagacaggagggtgttcattgaaagtgaaggagagttggtgatccagaagctgttgtatcatgagtgcctctgctctgagctccatcagattcccaggaagcatctctgagtaccttcaagtagcctctgtgctcagcctgctcctgctgctcttcaagacagcccagctctacctgcacaggcaatggctactcagcagtactcagcagttcccatccccaccttctcactggctctttggacacaagatcttaaaggaccaggaccttcaagatattctaactaggattaagaatttcccaagtgcctgtccacagtggctctggggaagcaaagtgcgcattcaagtgtatgaccctgactacatgaagctgattctggggagatcagacccaaaagctaatggttcctacagatttctagctccctggattgggcgtggtttgcttatgctggatggacagacatggtttcagcaccgacgaatgttgaccccagctttccactatgacattctgaagccttatacggaaatcatggcagactctgttcgtgtaatgctggataaatgggaacagattgttggccaggattccaccctggagatctttcgacacatcaccttgatgaccttggacaccatcatgaagtgtgccttcagccacgagggcagtgtccagttggacagaaaatacaagtcctatatccaggcagttgaggacctgaacgatctcgttttttcccgtgtgcggaacatctttcacctgaatgacatcatctacagagtgtcctctaatggctgcaaggctaacagtgcctgcaaacttgcccatgatcacacagaccaagtgatcaaatcaaggaggattcaacttcaggatgaggaagagttggaaaagcttaagaagaaaaggcgattggatttcctggacatcctcctatttgccagaatggaaaatggaaaaagcttatctgataaggaccttcgtgctgaagtggatactttcatgttcgagggccatgacaccacagctagtggtatctcctggatcttctatgctttggccacaaatcctgaacatcaacagagatgcaggaaggagatccaaagtctcctaggagatgggacttctatcacctggaatgacctggacaagatgccctatactaccatgtgcatcaaggaggccctgaggatctaccctcctgtaccaagtgtgagcagagagctcagctcacctgtcacctttccagatggacgttctttacccaaaggtatccatgttatgctctccttttatggccttcatcacaacccaactgtgtggccaaatccagaggtgtttgatccttctcgatttgcaccagggtcttcccggcacagccactcattcctgcccttctcaggaggagcaaggaactgcattgggaaacagtttgcgatgaatgagctgaaggtggctgtggccctgaccctgctccgctttgagctgctgccagatcccaccagagtcccaatccccataccaagaattgtgttgaagtccaagaatgggatccacttgcatctcaaagagctccaataatcttcacaggacaagacagctcaaatgcatgctgcctgccattctgtctttctgtcacttactcttttccccaatccttctgctcacatctcattctttcttctcaccttgttcacctccacccaccttctgctgggcttccagtctccttgcctgtcagtctttttcaacttcttctgagatccctacttgcttttctctctacctgtccctaaccagactgcatgtttgacctttgactttaatgatctccctaacttgcaccctgcctttcttttctgtgtatttccttctcttctactcttgtc

SEQ ID NO: 5 - protein, 519 aa, Artificial Sequence
  Description of Artificial Sequence; Note = synthetic construct
MSVSVLSPSRLLGDVSGILQAASLLILLLLLIKAVQLYLHRQWLLKALQQFPCPPSHWLFGHIQELQQDQELQRIQKWVETFPSACPHWLWGGKVRVQLYDPDYMKVILGRSDPKSHGSYRFLAPWIGYGLLLLNGQTWFQHRRMLTPAFHYDILKPYVGLMADSVRVMLDKWEELLGQDSPLEVFQHVSLMTLDTIMKCAFSHQGSIQVDRNSQSYIQAISDLNNLVFSRVRNAFHQNDTIYSLTSAGRWTHRACQLAHQHTDQVIQLRKAQLQKEGELEKIKRKRHLDFLDILLLAKMENGSILSDKDLRAEVDTFMFEGHDTTASGISWILYALATHPKHQERCREEIHSLLGDGASITWNHLDQMPYTTMCIKEALRLYPPVPGIGRELSTPVTFPDGRSLPKGIMVLLSIYGLHHNPKVWPNPEVFDPFRFAPGSAQHSHAFLPFSGGSRNCIGKQFAMNELKVATALTLLRFELLPDPTRIPIPIARLVLKSKNGIHLRLRRLPNPCEDKDQL

SEQ ID NO: 6 - DNA, 2576 bp, Artificial Sequence
  Description of Artificial Sequence; Note = synthetic construct
gaattccgcagagatccagcaggtgctgcaccatgagtgtctctgtgctgagccccagcagactcctgggtgatgtctctggaatcctccaagcggcctccctgctcattctgcttctgctgctgatcaaggcagttcagctctacctgcacaggcagtggctgctcaaagccctccagcagttcccgtgccctccctcccactggctcttcgggcacatccaggagctccaacaggaccaggagctacaacggattcagaaatgggtggagacattcccaagtgcctgtcctcattggctatggggaggcaaagttcgtgtccagctctatgaccctgactatatgaaggtgattctggggagatcagacccgaaatcccatggttcctacagattcctggctccatggattgggtacggcttgctcctgttgaatgggcagacatggttccagcatcgacggatgctgaccccagccttccactatgacatcctgaagccctatgtggggctcatggcagactctgtacgagtgatgctggacaaatgggaagagctccttggccaggattcccctctggaggtctttcagcacgtctccttgatgaccctggacaccatcatgaagtgtgccttcagccatcagggcagcatccaggtggacaggaattctcagtcctacatacaggccattagtgacctgaacaacctggttttttcccgtgtgaggaatgcctttcaccagaatgacaccatctacagcctgacctctgctggccgctggacacaccgcgcctgccagctggcccatcagcacacagaccaagtgatccaactgaggaaggctcaactacagaaggagggggagctggagaagatcaagaggaagaggcatttggattttctggatatcctcctcttggccaaaatggagaatgggagcatcttgtcagacaaggacctccgtgctgaggtggacacgttcatgtttgagggccacgacaccacagccagtgggatctcctggatcctctatgctctggccacacaccccaagcatcaggagaggtgccgggaggagatccacagcctcctgggtgatggagcctccatcacctggaaccacctggaccagatgccctacaccaccatgtgcattaaggaggcactgaggctctacccaccggtgccaggcattggcagagagctcagcactcccgtcaccttccctgatgggcgctccttgcccaaaggtatcatggtcctcctctccatttatggccttcaccacaacccaaaagtgtggcccaacccagaggtgtttgaccctttccgttttgcaccgggttctgctcaacacagccacgctttcctgcccttctcaggaggatcaaggaactgcattgggaaacaatttgccatgaacgagctgaaggtggccacggccctgaccctgctccgctttgagctgctgcctgatcccaccaggatccccatccccattgcacgacttgtgttgaaatccaaaaatggaatccacctgcgtctcaggaggctccctaacccttgtgaagacaaggaccagctttgagggcctccacctgccgtcctgtcttcctgacccccgcttctgtccccttcctgtctgcccatatcctgttttctgtctgcccaccttcccttcttcccacctgcctgctgtcccccagtctgcctgcccttctctctctcacctttctccaggctccctacctgcttgtctacctgtctcctacccacctgtatctcttgttgggagaaaagctgagtgttgggagaagctgaggccgagcttgcatgtctgacataatgtaaaagagtcttgaatcatgtccaggatccagggtctaaaaccccttgtggcctttggaacaccaagctctgtgctgaagggtggaaggctaccctgacgcaccataatctaagcccggggcataaaacccctcgtggcttggatagaatccagggctcgtggctctggaatgtgtctggacttgctggctccttgctccttgctctcccaggatcaattgtatcttgagttaaaagaacctgctctccattatctcaagtaacagagcagatgctaaaccgtcacagctgtaaattgtgtgcttaatgcaacatgccctttcgacccaccccccattctcaccacctgtttctttgtttgatcaccaataaataatctgcacttccagagctcggggccttcacagcctccatccttagctttggcgccctggacccactttctctctcaaactgtcttttctcactgctttgactctgccggactttgtcacccccacgacctggtgttgggtctgaacaccccaacatccctgaatctccacccacctcccaaactcctgcctgccctccagactgtctgcccatacacctgtctccttcttcctgcctggcttgtctgttcctatattagtttcctattactgctgtaataaactatcacaatctcagtgattttaaataaaaaaaaaaaaaaaaaaaaaaaaaaaaaaaaacggaattc

SEQ ID NO: 7 - protein, 519 aa, Artificial Sequence [sequence omitted]

SEQ ID NO: 8 - DNA, 1872 bp, Artificial Sequence
  Description of Artificial Sequence; Note = synthetic construct
tcaagtctgttttcctcgcagcggtgcccacacccctagcatactgcctggcacacagtaaatgctcattaaatatttgtgaggtacatggacaggggaatgtggtagacagctgatgaagctgttctcccactgttcccctaggtggatcatccaaagtcaatcgattctgaactctgaggtccaagttctgccctcccccttcactctccccacaagtgggcgggacaatcctcccatgacttaagcacaggtggacaggggtggtcagagagaggaaggggcactcagagatccagcaggtgctgcaccatgagtgtctctgtcctgagccccagcagacgcctgggtggtgtctccgggatcctccaagtgacctccctgctcattctgcttctgctgctgatcaaggcagctcagctctacctgcataggcagtggctgctcaaagccctccagcagttcccgtgccctccctcccactggctcttcgggcacatccaggagttccaacacgaccaggagctacaacggattcaggaacgggtgaagacattcccaagtgcctgtccttattggatatggggaggcaaagttcgtgtccagctctatgaccctgactatatgaaggtgattctggggagatcagacccgaaatcccatggttcctacagattcctggctccacggattgggtacggcttgctcctgttgaatgggcagacatggttccagcatcgacggatgctgaccccagccttccacaatgacatcctgaagccatatgtggggctcatggcagactctgtacgagtgatgctggacaaatgggaagagctccttggccaggattcccctctggaggtctttcagcacgtctccttgatgaccctggacaccatcatgaagagtgccttcagccatcagggcagcatccaggtggacaggaattctcagtcctacatccaggccattagtgacctgaacagcctggttttttgctgtatgaggaatgcctttcatgagaatgacaccatctacagcctgacctctgctggccgctggacacaccgcgcctgccagctggcccatcagcacacagaccaagtgatccaactgaggaaggctcaactacagaaggagggggagctggagaagatcaagaggaagaggcacttggattttctggacatcctcctcttggccaaaatggagaatgggagcatcttgtcagacaaggacctccgtgctgaggtggacacgttcatgtttgagggccacgacaccacagccagtgggatctcctggatcctctatgctctggccacacaccccaagcatcaggagaggtgccgggaggagatccatggcctcctgggtgatggagcctccatcacctggaaccacctggaccagatgccctacaccaccatgtgcattaaggaggcactgaggctctacccaccggtgccaggcattggaagagagctcagcactcccgtcaccttccctgatgggcgctccttgcccaaaggtatcatggtcctcctctccatttatggccttcaccacaacccaaaagtgtggcccaacctagaggtgtttgacccttcccgttttgcaccgggttctgctcaacacagccacgctttcctgcccttctcaggaggatcaaggaactgcatcgggaaacaatttgccatgaaccagctgaaggtggccagggccctgaccctgctccgctttgagctgctgcctgatcccaccaggatccccatccccattgcacgacttgtgttgaaatccaaaaatggaatccacctgcgtctcaggaggctccctaacccttgtgaagacaaggaccagctttga

SEQ ID NO: 9 - DNA, 21990 bp, Artificial Sequence
  Description of Artificial Sequence; Note = synthetic construct
nnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnggtcagagagaggtaggggcactcagagatccagcaggtgctgcaccatgagtgtctctgtgctgagccccagcagactcctgggtgatgtctctggaatcctccaagcggcctccctgctcattctgcttctgctgctgatcaaggcagttcagctctacctgcacaggcagtggctgctcaaagccctccagcagttcccgtgccctccctcccactggctcttcgggcacatccaggagnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnggatatggggaggcaaagttcgtgtccagctctatgaccctgactatatgaaggtgattctggggagatcaggtgagatcgaacccccatcccaactgcaatttctcttccctcttgacacatgcccctgggtctgtaaaattccagaagagagtggctgttcaaacatcaatatctgaaacctctcccacgcatccaccaagcccaccatgcccaggctgtagtcaaaaatatttatgaatactgaatgtttaaaacaatatgtggggctgaattttctattctttttttctatttctctattaagaaaattaaattttctgaaacgtaaattgccccagatctgtaaaacaaatttcatacccacattgacttgggttcgaagctctaattgttgcctgtcctggtcaggaggtgactaggaaaacatcaatgtagaaaaacccatggctctgtattattcattgtcattgaagacctagcctatgtatctcaccaattcaccttcctagcccctgggtgttttcctgctggaacactgagtctgtcttcctgagcccacattgaccttcaggacagaaatagaagtggatgggagtcaagtgggaggtgacatgggtcaaactgccaactgacagacaccaagaattgatgcagcctctaagactgccttcttagtaactcctagtttcctataaagccctttcttgcctttcagacccgaaatcccatggttcctacagattcctggctccatggattggtatgtgtgcaaactaggactgcagcccactccctagtcaggtttgagttatttatttggctcacagagaacaacagattagaggccaccactatcatgacctcatccccaaatcaagcctcacttccttcttctaacaagaccctcaccccttcctaatgctggtgcaaaccttcctgaggctcagcttcttccatttcatgtctcagttctctccagacatccttggcttcacatttattttacatctgcccacaacccttttcaacctttatctcacagctgtcataatcacatggatgtagcagctcaatcagaatattcacatgcctgaaatatacctccttttcctgcatttgcccatgacgtggcttcttctggatagttctccccttgagaagccacctttctccctcccaaattgtcaccagtcatccctagctccttgcagccnnnnnnnnnnnnnnnnnnnnnnnnnnnnnnnnnnncctgacacacacacacacacacacaaacacacattcatgtctctcttagggtacggcttgctcctgttgaatgggcagacatggttccagcatcgacggatgctgaccccagccttccactatgacatcctgaagccctatgtggggctcatggcagactctgtacgagtgatgctggtgagtccatgtctcnnnnnnnnnnnnnnnnnnnnnnnnnnnnnnnnnnnnnnnnnnnnnnnnnnnnnaaccctgtgtcccacaggcagacatagacatagacacatggaacaacactctcaggtcattgctgtgagagtagagggttccccagagttgtataaggtaggagaacacccaggcatcaggtcatatccacactttgttccccaccataggaatagagattcatggtgaacacaaggcccttctcctcccactttggaacctcagcacaagggactggaggcatatgcaatcttgttggacagtaggacttcctatgctggctggcctgggcaatggcagcttcagtggcagcatggacaggccaagatgtcacccacccaggctctggcctggggccccaggtttctgcatggatttaagccaccctgggaaggaaatgaacaccaggtctatgttctcagagcaataccttccatagatagcatcatctccagtcaggactctgatttctcacccaactgtgcccagcacattttgatgaatggaaaagaattgaagtccctgttttctctacaacacagtgnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnntgtattaaaagtgtgtgagaattaggcgtcagtttaccaacactggctttacagtctcagtttctacaatgcgtctgctctgtaaagtgaggaatcccaaccaagatatctgcaagctacaggaaaaaacaggatatctgcaggtacatgaaaaagccaggccttattagcaaagcacttcttggagattaagaaggtccatgccccctcccaccacaggacaaatgggaagagctccttggccaggattcccctctggaggtctttcagcacgtctccttgatgaccctggacaccatcatgaagtgtgccttcagccatcagggcagcatccaggtggacaggtcagtgacaaccctccagctgcagggcccttgttcttatcaagtggagtgcacatacctgaggggcaggtggggcagcgtgaaggctcaccgccacacaaaggaagagtcagtccctgaccaaactctaattccagtccagaccaaacaaagtctcaggaacagatgcctgattcctagcacaggtggaccctggatgttcataccatctgataaaggccaaaggataatagggctgtaagatagaagaaacattgcctcaaggctgcttgtccccattatccgaggctgcctcctctcagaccccattctacttccgggtaatgggcccacccctactgcagtcccttcttcatactctccctcaggaattctcagtcctacatacaggccattagtgacctgaacaacctggttttttcccgtgtgaggaatgcctttcaccagaatgacaccatctacagcctgacctctgctggccgctggacacaccgggcctgccagctggcccatcagcacacaggttctgtcttttcctcttgtctcccagcctttcccaggcacagcgaaagaacttgccctgactcctcaggcagagaaggcccctagtaaccctgcagaacgcagaacacactcagcctggggaattcccttgctcaggggctgggagctgagatgtgaggggccagattctgtgccttggtttatcaatgtccccacatggagataactgaatgagaccctgtccaaacagcattcaagaggagcctccatgaactgtgccactggcgggaggggtgccctgcctcacacagtactagagcttccacccctgaccctgcacccacactcacattcatgctgagatctaccaggcacccacactgggcagctaggcctcctgggggctgctgacggtctcagctctgcctggtaaccattgttctggtacagaccaagtgatccaactgaggaaggctcaactacagaaggagggggagctggagaagatcaagaggaagaggcatttggattttctggacatcctcctcttggccaaagtgagtatgtgtaggagaggcctgagtctttgcccagaagtacatagcaagagacaagccctgcactttcaccacgggtcttaccagatggagaatgggagcatcttgtcagacaaggacctccgtgctgaggtggacacgttcatgtttgagggccatgacaccacagccagtgggatctcctggatcctctatgctctggccacacaccccaagcatcaggagaggtgccgggaggagatccacagcctcctgggtgatggagcctccatcacctggtgagtgagggctcaaaagatggggttccctgccttctccacaggggcccctggtctgcccaggccttgctggtgttcaggatggaattgtttcaggaaccacctggaccagatgccctacaccaccagtgcataaggaagcactgaggctcaaccacctggaccagatgccctacaccaccatgtgcattaaggaggcactgaggctctccccaccggtgccaggcattggcagagagctcagcactcccgtcaccttccctgatgggcgctccttgcccaaaggtatgaagttcccccaccctctcacctaaactctccacagggacgtgtggagggtgagaaatccatgtgtgcatcagaattctgcacatctctgggtctcccttttgttctaaaaaaatcaaaaacatactttgtacttgggataaagaatttgaaaagtctggctgagagcttgaacccaccaaaagttcaagaaataaattttgatctctgaatgtggccttgggtcagtaactgtaaaattctattccctgagatgtgcagggctggaaagagaatcagacaagggcagagagggatgtgtttctttcctcatggggtcagtgcaaaagaggcttatcaggaatttcatttcctgggtcagctgttgtccagtctctgaggaaccctcaggttgatggcagagagaaagtgatgaccagatctggggacaccaaggtgcaactccctgggttctggtcccagtcctgccataacctagttgtgtgaacatagacaagacaattggtctctctgagattcaggtatctcccctgaaaactgagagcaaaagaatgtcttacttggagcgttgttgtactgagtgagttcatgtatattctccaatgacccttggaggatatnnnnnnnnnnnnnnnnnnnnnnnnnnnnnnnnnnnnnnnnnnnnnnnnnnnnnnnnnnnnnnnnnnnnnnnnnnnnnnaatgattgtctattcatcatctgaactcacatgctttgttaagcctaacccacctgcataatggcagaatcatcccagtcaaagtgaaaatttgtggaaagtaggctttttgggccttcttttgtgcctgctggctgaagtaccaggccaccccatgcaaatgattggtcttctgtctgtttccaacctgcaccacaggtatcatggtcctcctctccatttatggccttcaccacaacccaaaagtgtggcccaacccagaggtatgtggtccttgagaggaggaaatgaggtgatccctcaagaccaataccttctcctgcttccacctctgggagtcctgtcccccatgggtggcaagtaggtgctggatccttaactatcctggctctggtgctctctctgcaggtgtttgaccctttccgttttgcaccgggttctgctcaacacagccacgctttcctgcccttctcaggaggatcaaggtgagacgtcctgtgtggtaattcgaatagaggaatgagggaagtctctggtcaaccctctgatctttgtgagcctgatgttcatatgtggcatcttcaggtgtgctcttaaatgttggtatttgtgggaaagtcaggcacctggtgtgggcgtctctgtgtacaaaaggaaggtggcattcagagcaccccatggagactttgctccctctgcttcttcaaatgggcagcctgaattcactgtcagtgcatgttccaaacttcagtatattcatgtattttcctcactttaaggatatgaattctcaaaattagatattctccagtaaattcaacttcagctgtttgcttttctcagttgtcaggaatagtaaactgtagatttctctccccccacacatcctcaatgcagcacactaccttccctatttaattcactagtctgtcgtagagatgaatcattgaagtctttgcatctgtttataatacagatgacctgcaagtcctacctttcaagtcatataagggagaggttaaggtagccctttgaagaaagttttacaacagtgtgtgtcatatcatacatttttctgtcttgcccaatgacatttttactgcattatatagtgttcacatatttnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnncccactgcccttctgactccccttaatacacccatcggggcccctatgaggattcaggtccctgtgtgtcccactacatgggaagcacctgagagtgtggcccaccctcaccctacaggacctgaactgacctccacactggagaacaagaaatgtttgtggaggagtgaacaaaccattgattaactctttatctattcatgtgattagactctgtgccttagagccctggccttgtcttgatagagggtttcaggctgctgagagcagaggtagagggtaggagttaggagctagcccaggaagacggctctgtccaaggatcgggatagtgtgaagccaggacggggtagaagtgtccatgggctgtatgtgtgcaggggctggacacatgaggttgggcactgaatgtccagctcagggctggggtcaggggccaaaacctgctcagatcagaatggggcctgaggacacttctcaattcattatctccacctggcccaggaactgcatcgggaaacaatttgccatgaacgagctgaaggtggccacggccctgaccctgctccgctttgagctgctgcctgatcccaccaggatccccatccccattgcacgacttgtgttgaaatccaaaaatggaatccacctgcgtctcaggaggctccctaacccttgtgaagacaaggaccagctttgagggcctccacctgccgtcctgtcttcctgacccccgcttctgtccccttcctgtctgcccatatcctgttttctgtctgcccaccttcccttcttcccacctgcctgctgtcccccagtctgcctgcccttctctctctcacctttctccaggctccctacctgcttgtctacctgtctcctacccacctgtatctcttgttgggagaaaagctgagtgttgggagaagctgaggccgagcttgcatgtctgacataatgtaaaagagtcttgaatcatgtccaggatccagggtctaaaaccccttgtggcctttggaacaccaagctctgtgctgaagggtggaaggctaccctgacgcaccataatctaagcccggggcataaaacccctcgtgcttggatagaatccagggctcgtggctctggaatgtgtctggacttgctgcctcctcgctccttgctctcccaggatcaattgtatcttgagttaaaagaacctgctctccattatctcaagtagcagagcagatgctaaaccgtcacagctgtaaatcatgtggttaatgcaacatgccctttcgacccccacattctcaccacctgtttctttgtttgatcaccaataaataatctgcacttccagagctcggggccttcacagcctccatccttagctttggcgccctggacccactttctctctcaaactgtcttttctcactgctttgactctgccggactttgtcacccccacgacctggtgttgggtctgaacaccccaacatccctgaatctccacccacccnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnn

(19) United States
(12) Patent Application Publication
(10) Pub. No.: US 2002/0165208 A1
(43) Pub. Date: Nov. 7, 2002

(54) Compositions and methods relating to hypertension

(76) Inventors: Jorge H. Capdevila, Nashville, TN (US); Michael Waterman, Nashville, TN (US); Vijakumar Holla, Nashville, TN (US)

Correspondence Address: NEEDLE & ROSENBERG, P.C., The Candler Building, Suite 1200, 127 Peachtree Street, N.E., Atlanta, GA, 30303-1811, US

(21) Appl. No.: 09/942,429

(22) Filed: Aug. 29, 2001

Related U.S. Application Data
(60) Provisional application No. 60/228,947, filed on Aug. 29, 2000.

(51) Int. Cl. A61K 31/56

(52) U.S. Cl. 514/169

(57) ABSTRACT

This invention relates generally to compositions and methods relating to hypertension. The present invention provides a method of enhancing the activity of Cyp 4A14 by administering an agent thatenhances the activity of Cyp 4A14 as well as a method of inhibiting the activity of Cyp 4A14 by administering an agent that inhibits the activity of Cyp 4A14. Further provided, is a method of enhancing the activity of Cyp 4A12 by administering an agent that enhances the activity of Cyp 4A12 as well as a method of inhibiting the activity of Cyp 4A12 by administering an agent that inhibits the activity of Cyp 4A12. A method of enhancing the activity of Cyp 4A12 by administering an agent that inhibits the activity of Cyp 4A14 is also provided. The invention also provides a method of inhibiting the activity of Cyp 4A12 by administering an agent that enhances the activity of Cyp 4A14. Further provided is a method of enhancing the activity of human Cyp 4A11 by administering an agent that enhances the activity of human Cyp 4A11 as well as a method of inhibiting the activity of human Cyp 4A11 by administering an agent that inhibits the activity of human Cyp 4A11.

This application claims priority to U.S. provisional application Ser. No. 60/228,947 filed on Aug. 29, 2000. The 60/228,947 provisional patent application is herein incorporated by this reference in its entirety.

### BACKGROUND OF THE INVENTION

1. Field of the Invention

This invention relates generally to compositions and methods relating to hypertension.

### INTRODUCTION

Hypertension affects a significant proportion of the adult population of the western world and is a leading cause of cardiovascular disease and mortality. Gender differences in the prevalence and severity of the disease have suggested involvement of sex-dependent mechanisms in the pathogenesis of human hypertension (2,3) although the molecular basis of this association has remained poorly defined. Notwithstanding extensive efforts, the genetic bases of hypertension remain elusive and a lack of novel candidate genes continues to limit progress in this clinically important area of research.

Accordingly, it is clear that an understanding of the mechanisms that underlie hypertension is needed to improve the ability to identify drugs to be used in the treatment of hypertension, and to be used for the diagnosis of a predisposition to hypertension.

### SUMMARY OF THE INVENTION

In accordance with the purpose(s) of this invention, as embodied and broadly described herein, this invention, in one aspect, relates to a method of enhancing the activity of Cyp 4A14 by administering an agent that enhances the activity of Cyp 4A14.

The invention also relates to a method of inhibiting the activity of Cyp 4A14 by administering an agent that inhibits the activity of Cyp 4A14.

The invention also relates to a method of inhibiting the activity of testosterone by administering an agent that enhances the activity of Cyp 4A14.

The present invention also provides a method of enhancing the activity of testosterone by administering an agent that inhibits the activity of Cyp 4A14.

The invention also relates to a method of enhancing the activity of Cyp 4A12 by administering an agent that enhances the activity of Cyp 4A12.

Also provided herein is a method of inhibiting the activity of Cyp 4A12 by administering an agent that inhibits the activity of Cyp 4A12.

Further provided by the invention is a method of inhibiting the activity of Cyp 4A12 by administering an agent that inhibits the activity of testosterone.

The invention also relates to a method of enhancing the activity of Cyp 4A12 by administering an agent that enhances the activity of testosterone.

Also provided is a method of enhancing the activity of Cyp 4A12by administering an agent that inhibits the activity of Cyp 4A14.

Also provided is a method of inhibiting the activity of Cyp 4A12 by administering an agent that enhances the activity of Cyp 4A14.

Also provided is a method of enhancing the activity of human Cyp 4A11 by administering an agent that enhances the activity of human Cyp 4A11.

The invention also provides a method of inhibiting the activity of human Cyp 4A11 by administering an agent that inhibits the activity of human Cyp 4A11.

The invention also provides a method of enhancing the activity of human Cyp 4A11 by administering an agent that inhibits the activity of Cyp 4A14.

The invention also provides a method of inhibiting the activity of human Cyp 4A11 by administering an agent that enhances the activity of Cyp 4A14.

Also provided is a method of inhibiting the activity of human Cyp 4A11 by administering an agent that inhibits the activity of testosterone, as well as a method of enhancing the activity of human Cyp 4A11 by administering an agent that enhances the activity of testosterone.

The invention also provides a method of enhancing the activity of human Cyp 4A11 by administering an agent that inhibits the activity of Cyp 4A14.

The invention also provides a method of inhibiting the activity of human Cyp 4A11 by administering an agent that enhances the activity of Cyp 4A14.

Also provided is an isolated Cyp 4A14 polypeptide having the amino acid sequence of SEQ ID NO: 2, as well as an isolated Cyp 4A14 polynucleotide that encodes the amino acid sequence of SEQ ID NO: 2, and an isolated Cyp 4A14 polynucleotide having the nucleotide sequence of nucleotides 1637-4123 of SEQ ID NO: 1.

Also provided is an isolated Cyp 4A12 polypeptide having the amino acid sequence of SEQ ID NO: 3, as well as an isolated Cyp 4A12 polynucleotide that encodes the amino acid sequence of SEQ ID NO: 3, and an isolated Cyp 4A12 polynucleotide having the nucleotide sequence of nucleotides 282-2116 of SEQ ID NO: 4.

The invention further provides an isolated Cyp 4A11 polypeptide having the amino acid sequence of SEQ ID NO: 5, as well as an isolated Cyp 4A11 polynucleotide that encodes the amino acid sequence of SEQ ID NO: 5, and an isolated Cyp 4A11 polynucleotide having the nucleotide sequence of nucleotides 33-1589 of SEQ ID NO: 6. The present invention also provides a genomic sequence of Cyp 4A11 as set forth in SEQ ID NO: 9. Fragments of this genomic sequence are also contemplated by the present invention.

The invention also provides an isolated Cyp 4A22 polypeptide having the amino acid sequence of SEQ ID NO: 7, as well as an isolated Cyp 4A22 polynucleotide that encodes the amino acids sequence of SEQ ID NO: 7, and an isolated Cyp 4A22 polynucleotide having the nucleotide sequence of nucleotides 313-1869 of SEQ ID NO: 8 and a polynucleotide having the nucleotide sequence of nucleotides 313-1870 of SEQ ID NO: 8.

The invention also provides a method of identifying an agent capable of enhancing the activity of Cyp 4A14, comprising contacting Cyp 4A14 with a test agent, and determining if the activity of Cyp 4A14 is enhanced as compared to the activity of uncontacted Cyp 4A14, whereby an increase in Cyp4A14 activity indicates that the test agent is capable of enhancing the activity of Cyp 4A14.

The invention also provides a method of identifying an agent capable of inhibiting the activity of Cyp 4A14, comprising contacting Cyp 4A14 with a test agent, and determining if the activity of Cyp 4A14 is inhibited as compared to the activity of uncontacted Cyp 4A14, whereby a decrease in Cyp4A14 activity indicates that the test agent is capable of inhibiting the activity of Cyp 4A14.

The invention also provides a method of identifying an agent capable of enhancing the activity of Cyp 4A12, comprising contacting Cyp 4A12 with a test agent, and determining if the activity of Cyp 4A12 is enhanced as compared to the activity of uncontacted Cyp 4A12, whereby an increase in Cyp4A12 activity indicates that the test agent is capable of enhancing the activity of Cyp 4A12.

The invention also provides a method of identifying an agent capable of inhibiting the activity of Cyp 4A12, comprising contacting Cyp 4A12 with a test agent, and determining if the activity of Cyp 4A12 is inhibited as compared to the activity of uncontacted Cyp 4A12, whereby a decrease in Cyp4A12 activity indicates that the test agent is capable of inhibiting the activity of Cyp 4A12.

The invention also provides a method of screening for an agent capable of inhibiting the activating effect of testosterone on the activity of Cyp 4A12, comprising contacting Cyp 4A12 with a test agent in the presence of testosterone, and determining if the activity of Cyp 4A12 is inhibited as compared to the activity of Cyp 4A12 in the presence of testosterone but which has not been contacted with the test agent, whereby a decrease in Cyp 4A12 activity indicates that the test agent is capable of inhibiting the activating effect of testosterone on the activity of Cyp 4A12.

The invention also provides a method of screening for an agent capable of enhancing the activating effect of testosterone on the activity of Cyp 4A12, comprising contacting Cyp 4A12 with a test agent in the presence of testosterone, and determining if the activity of Cyp 4A12 is enhanced as compared to the activity of Cyp 4A12 in the presence of testosterone but which has not been contacted with the test agent, whereby an increase in Cyp4A12 activity indicates that the test agent is capable of enhancing the activating effect of testosterone on the activity of Cyp 4A12.

The invention also provides a method of screening for an agent capable of inhibiting the activating effect of testosterone on the activity of human Cyp 4A11, comprising contacting human Cyp 4A 11 with a test agent in the presence of testosterone, and determining if the activity of human Cyp 4A11 is inhibited as compared to the activity of human Cyp 4A11 in the presence of testosterone but which has not been contacted with the test agent, whereby a decrease in human Cyp 4A11 activity indicates that the test agent is capable of inhibiting the activating effect of testosterone on the activity of human Cyp 4A11.

The invention also provides a method of screening for an agent capable of enhancing the activating effect of testosterone on the activity of human Cyp 4A11, comprising contacting human Cyp 4A11 with a test agent in the presence of testosterone, and determining if the activity of human Cyp 4A11 is enhanced as compared to the activity of human Cyp 4A11 in the presence of testosterone but which has not been contacted with the test agent, whereby an increase in human Cyp 4A11 activity indicates that the test agent is capable of enhancing the activating effect of testosterone on the activity of human Cyp 4A11.

The invention also provides a non-human transgenic mammal comprising a gene encoding murine Cyp 4A14 which has been inactivated or completely deleted. The non-human transgenic mammal can be a mouse. In the transgenic mouse, the endogenous murine Cyp 4A12 gene has also been inactivated or completely deleted, and a copy of the human Cyp 4A11 gene has been introduced into the genome of the mouse and is active in the mouse. In another embodiment, the human Cyp 4A11 gene has been inactivated.

The invention also provides a non-human transgenic mammal comprising a gene encoding murine Cyp 4A12 which has been inactivated. The non-human transgenic mammal can be a mouse, rat, or rabbit.

The invention also provides a method of identifying an agent capable of reducing hypertension, comprising administering a test agent to a transgenic mouse comprising a gene encoding murine Cyp 4A14 which has been inactivated or completely deleted, and comparing the blood pressure of the mouse to the blood pressure of the same breed of mouse to which the test agent has not been administered, wherein a lower blood pressure in the first mouse as compared to the second the second mouse indicates that the test agent is capable of reducing hypertension.

The invention further provides a method of identifying an agent capable of reducing hypertension, comprising administering a test agent to a transgenic mouse comprising a gene encoding murine Cyp 4A14 which has been inactivated or completely deleted, and in which the endogenous murine Cyp 4A12 gene has also been inactivated or completely deleted, and into which a copy of the human Cyp 4A11 gene has been where the Cyp4A11 gene is active in the mouse, and comparing the blood pressure of the mouse to the blood pressure of the same breed of mouse to which the test agent has not been administered, wherein a lower blood pressure in the first mouse as compared to the second the second mouse indicates that the test agent is capable of reducing hypertension.

The invention also relates to a method of treating hypertension in an individual comprising inhibiting testosterone activity in the individual.

The invention also relates to a method of treating hypertension in an individual comprising enhancing Cyp 4A14 activity in the individual.

The invention also relates to a method of treating hypertension in an individual comprising inhibiting Cyp4A11 activity in the individual.

The invention also relates to a method of treating hypertension in an individual comprising inhibiting testosterone activity by enhancing 4A14 activity in the individual.

### BRIEF DESCRIPTION OF THE DRAWINGS

FIG. 1 shows the strategy used to construct the Cyp 4a14 pNTK targeting vector and for genotype analysis: shown are a partial restriction analysis and exon/intron distribution of the Cyp 4a14 gene and a pNTK targeting vector in which exons 10 and 11 are replaced by a neomycin resistance gene. Included is a Southern analysis of a HindIII digest of tail DNA by using the indicated 1.8-kb DNA probe.

FIG. 2 shows that the disruption of the Cyp 4a14 gene raises systemic blood pressures in a sexually dimorphic fashion: the blood pressures of conscious adult (10-to 14-week-old) male and female mice were measured by means of a right carotid artery catheter. Shown are averages±SE calculated from groups of 40 (−/−), 38 (+/+), or 12 (+/−) male mice (Top frame, A) or from groups of 20 (−/−) or 14 (+/+) female mice (Bottom frame, B). [Pressure differentials between Cyp 4a14(+/+) and (−/−) mice were of 38, 30, and 25 mm and of 14, 17, and 11 mm Hg, for mean, systolic, and diastolic blood pressures, and for male and female mice, respectively]. (A) Significantly differentfrom the male wild type: *, Cyp (+/−), P≧0.007; ** Cyp (−/−), P≧1×10⁻⁵. (B) Significantly different from the female wild type: *, Cyp (−/−), P≧1×10⁻⁴. No significant pressure differences were observed between female Cyp 4a14 (+/−) and 4a14 (+/+) mice.

FIG. 3 shows that hypertension in Cyp 4a14 (−/−) mice is androgen-sensitive: (Top frame, A): Groups of Cyp 4a14 (+/+) and (−/−) adult mice were castrated, and 10-12 days later their systemic blood pressures, as well as those of noncastrated knockout and wild-type mice, were determined. (Bottom frame, B): Groups of Cyp 4a14 (−/−) mice were castrated and implanted with either placebo (PL) or TST- or DHT-releasing pellets and their blood pressures, and those of noncastrated knockout controls, determined 9 days later (B). Shown are averages±SE calculated from groups of 38 4a14 (+/+), 40 4a14 (−/−), 4 castrated 4a14 (+/+), 16 castrated 4a14 (−/−), or from a group of 30 castrated 4a14 (−/−) mice treated with either placebo (8 mice) (CST/PL), TST (14 mice) (CST/TST), or DHT-releasing pellets (8 mice) (CST/DHT). FIG. 3A: Significantly different from the MABPs of control (+/+), castrated (+/+), and castrated (/) mice: *, P≧1×10⁻⁵. The MABP of castrated wild-type and knockout mice were not significantly different from that of wild type. (B) Significantly different from the MABP of castrated placebo Cyp (−/−) mice: *, P≧1×10⁻⁴; P≧1×10⁻⁵. The MABPs of control, castrated, and TST- or DHT-treated Cyp 4a14 (−/−) mice did not differ significantly.

FIG. 4 shows nucleic acid and in situ hybridization analysis of RNAs present in kidneys of control and DHT-treated adult mice. Top frame: Samples of total RNA (5-10 μg each) from the kidneys of control (A), castrated (B), castrated and DHT-treated (C) males or from control (D) and DHT-treated (E) females were fractionated by agar electrophoresis, transferred to nitrocellulose membranes, and hybridized to ³²P-labeled DNA probes (400-500 bp) coding for segments of the 3′-untranslated end of the Cyp 4a10, 4a12, and 4a14 cDNAs. After high-stringency washes, the membranes were exposed to x-ray films for 4, 2, or 21 h for male Cyp 4a10, 4a12, and 4a14, respectively, and 6, 21, or 12 h for female Cyp 4a10, 4a12, and 4a14, respectively. RNA loadings were normalized by using a -actin cDNA probe. Animal treatment protocols were as in FIG. 2 and Table 2. Long exposures revealed the presence of Cyp 4a14 reactive transcripts in 4a14 (−/−) mice kidneys (for example, lanes A-C). Reverse transcription-PCR amplification, cDNA cloning, and sequence analysis demonstrated that these were truncated mRNAs lacking exons ^{x} and XI, transcribed from the disrupted Cyp 4a14 gene. Bottom frame: Dehydrated paraffin sections from the kidneys of control (A and A′), castrated (B and B′) and DHT-treated castrated male mice (C and C′) were hybridized to [³⁵S]-labeled riboprobes encoding 3′-end untranslated segments of the Cyp 4a12 cDNA. After washing, RNase A treatment, and dehydration, the sections were dipped in emulsion (IlfordK5; Knutsford, Cheshire, U.K.), exposed for 4-5 days at 4° C., and developed by using D-19 (Kodak). Slides were counterstained with hematoxylin. Photomicrographs were obtained by using either dark-field (3×) (A-C) or bright-field (100×) (A′, B′, and C′) optics. Thick ascending limbs, collecting ducts, glomeruli, and vessels (v) are indicated by arrows t, c, g, and v, respectively.

FIG. 5 shows the impaired afferent arteriolar autoregulatory capacity in male Cyp 4a14 (−/−) mice. Kidneys from adult Cyp 4a14 (+/+) (n=6 mice; n=10 vessels) and (−/−) mice (n=5 mice; n=10 vessels) were perfused as described in the Examples, and the effects of changes in perfusion pressure on the diameter of the afferent arterioles were monitored by videomicroscopy. Values (in percentage of control diameter) are the mean±SEM. *, significant difference from diameter measured at 80 mm Hg in the same group (P<0.05). Control afferent arteriole diameters (at 80 mm Hg) were 19±0.5 and 17±0.4 μm for Cyp 4a14 (+/+) and (−/−), respectively. P≧0.05; n=10 vessels.

### DETAILED DESCRIPTION OF THE INVENTION

The present invention may be understood more readily by reference to the following detailed description of the invention and the Examples included therein and to the Figures and their previous and following description.

As used in the specification and the appended claims, the singular forms “a,” “an” and “the” include plural referents unless the context clearly dictates otherwise.

Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

The present invention relates to the surprising discovery that certain cytochrome P450 isoforms in mammals are involved in the regulation of systemic blood pressure in a sexually dimorphic manner. More specifically, it has been discovered that one cytochrome P450 (Cyp) isoform, Cyp 4A14, is involved in the regulation of testosterone expression, and that inactiviation of Cyp 4A14 an a transgenic mouse leads to an increase in testosterone activity, which in turn leads to an increase in the activity of another Cyp isoform, Cyp 4A12. The increase in Cyp 4A12 activity leads to the increased production of 20-hydroxyarachidonic acid (20-HETE or 20-OH-AA), which is known to have a vasoconstrictive activity in vitro, and results in a hypertensive phenotype.

As used herein, unless otherwise specified, an “increase in activity” or “enhanced activity” is defined as an increase in gene expression (such as an increase in expression of Cyp4A12), an increase in production (such as of the steroid testosterone), or an increase in the activity of the molecule, which includes but is not limited to, an increase in enzymatic activity or an increase in binding, such as binding of a molecule to nucleic acid or protein.

As used herein, unless otherwise specified, a “decrease in activity” or “inhibition” is defined as a decrease in gene expression (such as a decrease in expression of Cyp4A12), a decrease in production (such as of the steroid testosterone), or a decrease in the activity of the molecule, which includes but is not limited to, a decrease in enzymatic activity or a decrease in binding, such as binding of a molecule to nucleic acid or protein.

As used herein, reference to various Cyp polypeptides is intended to refer to the specific isoform named, as well as to functional homologs of that isoform in another species. Thus, for example, Cyp 4A12 is a murine Cyp isoform; references made herein to Cyp 4A12 are intended to encompass, for example, the human functional equivalent of murine Cyp 4A12, which is Cyp 4A11.

Accordingly, the invention, in one aspect, relates to a method of enhancing the activity of Cyp 4A14 by administering an agent that enhances the activity of Cyp 4A14.

An agent that enhances the activity of a Cyp 4A14 is defined as a compound that binds a Cyp 4A14 or a compound, including antibodies, that binds the target for Cyp 4A14 and enhances an activity of Cyp 4A14. The enhancing agent can be an antibody, either polyclonal or monoclonal, that specifically binds to Cyp 4A14, a ligand that binds to Cyp 4A14, a polypeptide that binds to Cyp 4A14 or a compound that binds to Cyp 4A14. Anti-idiotypic antibodies and affinity matured antibodies are also considered. Other agents that can enhance Cyp 4A14 activity include, but are not limited to molecules or compounds designed to enhance Cyp 4A14 activity. The enhancing agent can be a whole protein or a fragment of a protein that enhances Cyp 4A14 activity. Crystal structures of Cyp 4A14 may be utilized to design molecules that enhance Cyp 4A14 activity. An agent that enhances the activity of Cyp 4A14 can also be a compound, such as an antibody, a protein, a chemical, or another molecule that binds to regulatory regions of the Cyp 4A 14 gene and increases gene expression.

The invention also relates to a method of inhibiting the activity of Cyp 4A14 by administering an agent that inhibits the activity of Cyp 4A14.

An “inhibitor of Cyp 4A14” or an agent that inhibits the activity of Cyp4A14 is defined as a compound that binds Cyp 4A14 or a compound, including antibodies, that binds the target for Cyp 4A14 and prevents an activity of Cyp 4A14. The inhibitor can be an antibody, either polyclonal or monoclonal, that specifically binds to Cyp 4A14, a ligand that binds to Cyp 4A14, a polypeptide that binds to Cyp 4A14, a compound that binds to Cyp 4A14 or a peptide mimetic based on Cyp 4A14. Anti-idiotypic antibodies and affinity matured antibodies are also considered. Other inhibitors include, but are not limited to molecules or compounds designed to block Cyp 4A14 activity. The inhibitor can be a whole protein or a fragment of a protein that inhibits Cyp 4A14. Crystal structures of the Cyp 4A14 may be utilized to design molecules that inhibit Cyp 4A14 activity. An agent that inhibits the activity of Cyp 4A14 can also be a compound such as an antibody, a protein, a chemical, or another molecule that binds to regulatory regions of the Cyp 4A14 gene and inhibits gene expression.

The invention also relates to a method of inhibiting the activity of testosterone by administering an agent that enhances the activity of Cyp 4A14.

Also provided by this invention is a method of enhancing the activity of testosterone by administering an agent that inhibits the activity of Cyp 4A14.

The invention also relates to a method of enhancing the activity of Cyp 4A12 by administering an agent that enhances the activity of Cyp 4A12.

An agent that enhances the activity of a Cyp 4A12 is defined as a compound that binds a Cyp 4A12 or a compound, including antibodies, that binds the target for Cyp 4A12 and enhances an activity of Cyp 4A12. The enhancing agent can be an antibody, either polyclonal or monoclonal, that specifically binds to Cyp 4A12, a ligand that binds to Cyp 4A12, a polypeptide that binds to Cyp 4A12 or a compound that binds to Cyp 4A12. Anti-idiotypic antibodies and affinity matured antibodies are also considered. Other agents that can enhance Cyp 4A12 activity include, but are not limited to molecules or compounds designed to enhance Cyp 4A12 activity. The enhancing agent can be a whole protein or a fragment of a protein that enhances Cyp 4A12 activity. Crystal structures of Cyp 4A12 may be utilized to design molecules that enhance Cyp 4A12 activity. An agent that enhances the activity of Cyp 4A12 can also be a compound, such as an antibody, a protein, a chemical, or another molecule that binds to regulatory regions of the Cyp 4A12 gene and increases gene expression.

Also provided herein is a method of inhibiting the activity of Cyp 4A12 by administering an agent that inhibits the activity of Cyp 4A12.

An “inhibitor of Cyp 4A12” or an agent that inhibits the activity of Cyp 4A12 is defined as a compound that binds Cyp 4A12 or a compound, including antibodies, that binds the target for Cyp 4A12 and prevents an activity of Cyp 4A12. The inhibitor can be an antibody, either polyclonal or monoclonal, that specifically binds to Cyp 4A12, a ligand that binds to Cyp 4A12, a polypeptide that binds to Cyp 4A12, a compound that binds to Cyp 4A12 or a peptide mimetic based on Cyp 4A12. Anti-idiotypic antibodies and affinity matured antibodies are also considered. Other inhibitors include, but are not limited to molecules or compounds designed to block Cyp 4A12 activity. The inhibitor can be a whole protein or a fragment of a protein that inhibits Cyp 4A12. Crystal structures of the Cyp 4A12 may be utilized to design molecules that inhibit Cyp 4A12 activity. An agent that inhibits the activity of Cyp 4A12 can also be a compound such as an antibody, a protein, a chemical, or another molecule that binds to regulatory regions of the Cyp 4A12 gene and inhibits gene expression.

The present invention also relates to a method of inhibiting the activity of Cyp 4A12 by administering an agent that inhibits the activity of testosterone.

The invention also relates to a method of enhancing the activity of Cyp 4A12 by administering an agent that enhances the activity of testosterone.

Also provided is a method of enhancing the activity of Cyp 4A12 by administering an agent that inhibits the activity of Cyp 4A14.

Also provided is a method of inhibiting the activity of Cyp 4A12 by administering an agent that enhances the activity of Cyp 4A14.

Also provided is a method of enhancing the activity of human Cyp 4A11 by administering an agent that enhances the activity of human Cyp 4A11.

An agent that enhances the activity of a Cyp 4A11 is defined as a compound that binds a Cyp 4A11 or a compound, including antibodies, that binds the target for Cyp 4A11 and enhances an activity of Cyp 4A11. The enhancing agent can be an antibody, either polyclonal or monoclonal, that specifically binds to Cyp 4A11, a ligand that binds to Cyp 4A11, a polypeptide that binds to Cyp 4A11 or a compound that binds to Cyp 4A11. Anti-idiotypic antibodies and affinity matured antibodies are also considered. Other agents that can enhance Cyp 4A11 activity include, but are not limited to molecules or compounds designed to enhance Cyp 4A11 activity. The enhancing agent can be a whole protein or a fragment of a protein that enhances Cyp 4A11 activity. Crystal structures of Cyp 4A11 may be utilized to design molecules that enhance Cyp 4A11 activity. An agent that enhances the activity of Cyp 4A11 can also be a compound, such as an antibody, a protein, a chemical, or another molecule that binds to regulatory regions of the Cyp 4A11 gene and increases gene expression

The invention also provides a method of inhibiting the activity of human Cyp 4A11 by administering an agent that inhibits the activity of human Cyp 4A11.

An “inhibitor of Cyp 4A11” or an agent that inhibits the activity of Cyp 4A11 is defined as a compound that binds Cyp 4A11 or a compound, including antibodies, that binds the target for Cyp 4A11 and prevents an activity of Cyp 4A11. The inhibitor can be an antibody, either polyclonal or monoclonal, that specifically binds to Cyp 4A11, a ligand that binds to Cyp 4A11, a polypeptide that binds to Cyp 4A11, a compound that binds to Cyp 4A11 or a peptide mimetic based on Cyp 4A11. Anti-idiotypic antibodies and affinity matured antibodies are also considered. Other inhibitors include, but are not limited to molecules or compounds designed to block Cyp 4A11 activity. The inhibitor can be a whole protein or a fragment of a protein that inhibits Cyp 4A11. Crystal structures of the Cyp 4A11 may be utilized to design molecules that inhibit Cyp 4A11 activity. An agent that inhibits the activity of Cyp 4A11 can also be a compound such as an antibody, a protein, a chemical, or another molecule that binds to regulatory regions of the Cyp 4A11 gene and inhibits gene expression.

The invention also provides a method of enhancing the activity of human Cyp 4A11 by administering an agent that inhibits the activity of Cyp 4A14.

The invention also provides a method of inhibiting the activity of human Cyp 4A11 by administering an agent that enhances the activity of Cyp 4A14.

Also provided is a method of inhibiting the activity of human Cyp 4A11 by administering an agent that inhibits the activity of testosterone, as well as a method of enhancing the activity of human Cyp 4A11 by administering an agent that enhances the activity of testosterone.

The invention also provides a method of enhancing the activity of human Cyp 4A11 by administering an agent that inhibits the activity of Cyp 4A14.

The invention also provides a method of inhibiting the activity of human Cyp 4A11 by administering an agent that enhances the activity of Cyp 4A14.

Also provided by the present invention is an isolated Cyp 4A14 polynucleotide having the nucleotide sequence of nucleotides 1637-4123 of SEQ ID NO: 1. Nucleotides 1637-4123 of SEQ ID NO: 1 encode murine Cyp 4A14. SEQ ID NO: 1 also comprises nucleotide sequences upstream of the ATG start site (nucleotides 1-1636). The invention also provides an isolated Cyp 4A14 polynucleotide having the nucleotide sequence of nucleotides 1637-3157 of SEQ ID NO: 1. Further provided by the present invention is an isolated Cyp 4A14 polynucleotide that encodes the amino acid sequence of SEQ ID NO: 2. SEQ ID NO: 2 is the amino acid sequence of murine Cyp 4A14. The amino acid sequence of Cyp 4A14 and a nucleotide sequence encoding Cyp 4A14 can be accessed on GenBank via Accession No. NM 007822. The present invention also provides an isolated Cyp 4A14 polynucleotide having the nucleotide sequence of nucleotides 27-1550 of GenBank Accession No. NM 007822.

Also provided is an isolated Cyp 4A12 polynucleotide that encodes the amino acid sequence of SEQ ID NO: 3. SEQ ID NO: 3 is the amino acid sequence of murine Cyp 4A12. Further provided is an isolated Cyp 4A12 polynucleotide having the nucleotide sequence of nucleotides 282-2116 of SEQ ID NO: 4 and a polynucleotide having the nucleotide sequence of nucleotides 282-1805. SEQ ID NO: 4 also comprises nucleotide sequences upstream of the ATG start site (nucleotides 1-281).

The invention further provides an isolated Cyp 4A11 polynucleotide that encodes the amino acid sequence of SEQ ID NO: 5. SEQ ID NO: 5 is the amino acid sequence of human Cyp 4A11. The invention also provides an isolated Cyp 4A11 polynucleotide having the nucleotide sequence of nucleotides 33-2576 of SEQ ID NO: 6 and a polynucleotide having the nucleotide sequence of nucletoides 33-1589 of Seq ID NO: 6. SEQ ID NO: 6 also comprises nucleotide sequences upstream of the ATG start site (nucleotides 1-32). The amino acid sequence of human Cyp 4A11 and a nucleotide sequence encoding the amino acid sequence of human Cyp 411 can be found in GenBank via Accession No. S67580.

The invention further provides an isolated Cyp 4A22 polynucleotide that encodes the amino acid sequence of SEQ ID NO: 7, an isolated Cyp 4A22 polynucleotide having the nucleotide sequence of SEQ ID NO: 8, an isolated Cyp 4A22 polynucleotide having the nucleotide sequence of nucleotides 313-1869 of SEQ ID NO: 8 and a polynucleotide having the nucleotide sequence of nucleotides 313-1870 of SEQ ID NO: 8. The invention also provides a polynucleotide having nucleotides 1-312 of SEQ ID NO: 8. Cyp 4A22 is an isoform of Cyp 4A11. The genomic sequence of Cyp 4A22 is available from GenBank via Accession Number AF208532. The recombinant form of Cyp 4A22 is unable to catalyze arachidonic acid metabolism and 20-HETE formation. However, Cyp 4A11, an active arachidonate hydroxylase, shows 95% amino acid sequence identity with Cyp 4A22. There are a total of 24 amino acid differences between Cyp 4A11 and Cyp 4A22. Of these 24 differences, 4 differences can be classified as nonconservative substitutions. Therefore, the present invention also provides an isolated Cyp 4A22 polynucleotide containing mutations that convert Cyp 4A22 into an active arachidonic acid omega hydroxylase and/or 20-HETE synthase. The present invention also provides polynucleotides encoding a Cyp 4A22 variant that is capable of arachidonic acid hydroxylation. Fragments of Cyp 4A22 polynucleotides that encode Cyp 4A22 polypeptides that are capable of arachidonic acid hydroxylation are also contemplated by the present invention. One of skill in the art would know how to make mutations in the Cyp 4A22 nucleotide sequence and test the resulting polypeptide encoded by the mutated polynucleotide for arachadonic acid hydroxylation activity and/or 20-HETE synthase activity according to the teachings provided in the Examples and in the art.

As used herein, the term “polynucleotide” or “nucleic acid” refers to single-or multiple stranded molecules which may be DNA or RNA, or any combination thereof, including modifications to those nucleic acids. The nucleic acid may represent a coding strand or its complement, or any combination thereof. Nucleic acids may be identical in sequence to the sequences which are naturally occurring for any of the novel genes discussed herein or may include alternative codons which encode the same amino acid as that which is found in the naturally occurring sequence. These nucleic acids can also be modified from their typical structure. Such modifications include, but are not limited to, methylated nucleic acids, the substitution of a non-bridging oxygen on the phosphate residue with either a sulfur (yielding phosphorothioate deoxynucleotides), selenium (yielding phosphorselenoate deoxynucleotides), or methyl groups (yielding methylphosphonate deoxynucleotides).

A nucleic acid molecule encoding Cyp 4A14, Cyp 4A12, Cyp 4A11 or Cyp 4A22 can be isolated from the organism in which it is normally found. For example, a genomic DNA or cDNA library can be constructed and screened for the presence of the nucleic acid of interest. Methods of constructing and screening such libraries are well known in the art and kits for performing the construction and screening steps are commercially available (for example, Stratagene Cloning Systems, La Jolla, Calif.). Once isolated, the nucleic acid can be directly cloned into an appropriate vector, or if necessary, be modified to facilitate the subsequent cloning steps. Such modification steps are routine, an example of which is the addition of oligonucleotide linkers which contain restriction sites to the tennini of the nucleic acid. General methods are set forth in Sambrook et al., “Molecular Cloning, a Laboratory Manual,” Cold Spring Harbor Laboratory Press (1989).

Once the nucleic acid sequence of the desired Cyp polypeptide is obtained, the sequence encoding specific amino acids can be modified or changed at any particular amino acid position by techniques well known in the art. For example, PCR primers can be designed which span the amino acid position or positions and which can substitute any amino acid for another amino acid. Then a nucleic acid can be amplified and inserted into the wild-type Cyp coding sequence in order to obtain any of a number of possible combinations of amino acids at any position of the Cyp polypeptide. Alternatively, one skilled in the art can introduce specific mutations at any point in a particular nucleic acid sequence through techniques for point mutagenesis. General methods are set forth in Smith, M. “In vitro mutagenesis” Ann. Rev. Gen., 19:423-462 (1985) and Zoller, M. J. “New molecular biology methods for protein engineering” Curr. Opin. Struct. Biol., 1:605-610 (1991). Techniques such as these can be used to alter the coding sequence without altering the amino acid sequence that is encoded. Naturally occurring variants of the Cyp polypeptides of this invention are also contemplated herein. An example of a naturally occurring variant is a human Cyp 4A11 polypeptide, wherein tryptophan at position 126 of SEQ ID NO: 5 is substituted with arginine (Cyp 4A11/W126→R). Another example is a human Cyp 4A11 polypeptide, wherein arginine at position 231 of SEQ ID NO: 5 is substituted with cysteine (Cyp 4A11/R231→C). Further provided is a human Cyp 4A11 polypeptide, wherein methionine at position 369 of SEQ ID NO: 5 is substituted with arginine (Cyp 4A11/M369→R). Also provided is a human Cyp 4A11 polypeptide, wherein leucine at position 509 of SEQ ID NO: 5 is substituted with phenylalanine (Cyp 4A11/L509→F).

Another example of a method of obtaining a DNA molecule encoding a Cyp polypeptide is to synthesize a recombinant DNA molecule which encodes the Cyp polypeptide. For example, oligonucleotide synthesis procedures are routine in the art and oligonucleotides coding for a particular protein region are readily obtainable through automated DNA synthesis. A nucleic acid for one strand of a double-stranded molecule can be synthesized and hybridized to its complementary strand. One can design these oligonucleotides such that the resulting double-stranded molecule has either internal restriction sites or appropriate 5′ or 3′ overhangs at the termini for cloning into an appropriate vector. Double-stranded molecules coding for relatively large proteins can readily be synthesized by first constructing several different double-stranded molecules that code for particular regions of the protein, followed by ligating these DNA molecules together. For example, Cunningham, et al., “Receptor and Antibody Epitopes in Human Growth Hormone Identified by Homolog-Scanning Mutagenesis,” Science, 243:1330-1336 (1989), have constructed a synthetic gene encoding the human growth hormone gene by first constructing overlapping and complementary synthetic oligonucleotides and ligating these fragments together. See also, Ferretti, et al., Proc. Nat. Acad. Sci. 82:599-603 (1986), wherein synthesis of a 1057 base pair synthetic bovine rhodopsin gene from synthetic oligonucleotides is disclosed. By constructing a Cyp polyeptide in this manner, one skilled in the art can readily obtain any particular Cyp polypeptide with desired amino acids at any particular position or positions within the Cyp polypepitde. See also, U.S. Pat. No. 5,503,995 which describes an enzyme template reaction method of making synthetic genes. Techniques such as this are routine in the art and are well documented. These nucleic acids or fragments of a nucleic acid encoding a Cyp polypeptide can then be expressed in vivo or in vitro as discussed below.

The invention also provides for the isolated nucleic acids encoding a Cyp polypeptide in a vector suitable for expressing the nucleic acid. Once a nucleic acid encoding a particular Cyp polypeptide of interest, or a region of that nucleic acid, is constructed, modified, or isolated, that nucleic acid can then be cloned into an appropriate vector, which can direct the in vivo or in vitro synthesis of that wild-type and/or modified Cyp polypeptide. The vector is contemplated to have the necessary functional elements that direct and regulate transcription of the inserted gene, or nucleic acid. These functional elements include, but are not limited to, a promoter, regions upstream or downstream of the promoter, such as enhancers that may regulate the transcriptional activity of the promoter, an origin of replication, appropriate restriction sites to facilitate cloning of inserts adjacent to the promoter, antibiotic resistance genes or other markers which can serve to select for cells containing the vector or the vector containing the insert, RNA splice junctions, a transcription termination region, or any other region which may serve to facilitate the expression of the inserted gene or hybrid gene. (See generally, Sambrook et al.).

There are numerous E. coli (Escherichia coli) expression vectors known to one of ordinary skill in the art which are useful for the expression of the nucleic acid insert. Other microbial hosts suitable for use include bacilli, such as Bacillus subtilis, and other enterobacteriaceae, such as Salmonella, Serratia, and various Pseudomonas species. In these prokaryotic hosts one can also make expression vectors, which will typically contain expression control sequences compatible with the host cell (e.g., an origin of replication). In addition, any number of a variety of well-known promoters will be present, such as the lactose promoter system, a tryptophan (Trp) promoter system, a beta-lactamase promoter system, or a promoter system from phage lambda. The promoters will typically control expression, optionally with an operator sequence, and have ribosome binding site sequences for example, for initiating and completing transcription and translation. If necessary, an amino terminal methionine can be provided by insertion of a Met codon 5′ and in-frame with the downstream nucleic acid insert. Also, the carboxy-terminal extension of the nucleic acid insert can be removed using standard oligonucleotide mutagenesis procedures.

Additionally, yeast expression can be used. There are several advantages to yeast expression systems. First, evidence exists that proteins produced in a yeast secretion systems exhibit correct disulfide pairing. Second, post-translational glycosylation is efficiently carried out by yeast secretory systems. The Saccharomyces cerevisiae pre-pro-alpha-factor leader region (encoded by the MF″-1 gene) is routinely used to direct protein secretion from yeast. (Brake, et al., Alpha-Factor-Directed Synthesis and Secretion of Mature Foreign Proteins in Saccharomyces cerevisiae. Proc. Nat. Acad. Sci., 81:4642-4646 (1984)). The leader region of pre-pro-alpha-factor contains a signal peptide and a pro-segment which includes a recognition sequence for a yeast protease encoded by the KEX2 gene: this enzyme cleaves the precursor protein on the carboxyl side of a Lys-Arg dipeptide cleavage signal sequence. The nucleic acid coding sequence can be fused in-frame to the pre-pro-alpha-factor leader region. This construct is then put under the control of a strong transcription promoter, such as the alcohol dehydrogenase I promoter or a glycolytic promoter. The nucleic acid coding sequence is followed by a translation termination codon which is followed by transcription termination signals. Alternatively, the nucleic acid coding sequences can be fused to a second protein coding sequence, such as Sj26 or β- galactosidase, used to facilitate purification of the fusion protein by affinity chromatography. The insertion of protease cleavage sites to separate the components of the fusion protein is applicable to constructs used for expression in yeast. Efficient post translational glycosylation and expression of recombinant proteins can also be achieved in Baculovirus systems.

Mammalian cells permit the expression of proteins in an environment that favors important post-translational modifications such as folding and cysteine pairing, addition of complex carbohydrate structures, and secretion of active protein. Vectors useful for the expression of active proteins in mammalian cells are characterized by insertion of the protein coding sequence between a strong viral promoter and a polyadenylation signal. The vectors can contain genes conferring hygromycin resistance, genticin or G418 resistance, or other genes or phenotypes suitable for use as selectable markers, or methotrexate resistance for gene amplification. The chimeric protein coding sequence can be introduced into a Chinese hamster ovary (CHO) cell line using a methotrexate resistance-encoding vector, or other cell lines using suitable selection markers. Presence of the vector DNA in transformed cells can be confirmed by Southern blot analysis. Production of RNA corresponding to the insert coding sequence can be confirmed by Northern blot analysis. A number of other suitable host cell lines capable of secreting intact human proteins have been developed in the art, and include the CHO cell lines, HeLa cells, myeloma cell lines, Jurkat cells, etc. Expression vectors for these cells can include expression control sequences, such as an origin of replication, a promoter, an enhancer, and necessary information processing sites, such as ribosome binding sites, RNA splice sites, polyadenylation sites, and transcriptional terminator sequences. Preferred expression control sequences are promoters derived from immunoglobulin genes, SV40, Adenovirus, Bovine Papilloma Virus, etc. The vectors containing the nucleic acid segments of interest can be transferred into the host cell by well-known methods, which vary depending on the type of cellular host. For example, calcium chloride transformation is commonly utilized for prokaryotic cells, whereas calcium phosphate, DEAE dextran, or lipofectin mediated transfection or electroporation may be used for other eukaryotic cellular hosts.

Alternative vectors for the expression of genes or nucleic acids in mammalian cells, those similar to those developed for the expression of human gamma-interferon, tissue plasminogen activator, clotting Factor VIII, hepatitis B virus surface antigen, protease Nexinl, and eosinophil major basic protein, can be employed. Further, the vector can include CMV promoter sequences and a polyadenylation signal available for expression of inserted nucleic acids in mammalian cells (such as COS-7).

Insect cells also permit the expression of mammalian proteins. Recombinant proteins produced in insect cells with baculovirus vectors undergo post-translational modifications similar to that of wild-type proteins. Briefly, baculovirus vectors useful for the expression of active proteins in insect cells are characterized by insertion of the protein coding sequence downstream of the Autographica californica nuclear polyhedrosis virus (AcNPV) promoter for the gene encoding polyhedrin, the major occlusion protein. Cultured insect cells such as Spodoptera frugiperda cell lines are transfected with a mixture of viral and plasmid DNAs and the viral progeny are plated. Deletion or insertional inactivation of the polyhedrin gene results in the production of occlusion negative viruses which form plaques that are distinctively different from those of wild-type occlusion positive viruses. These distinctive plaque morphologies allow visual screening for recombinant viruses in which the AcNPV gene has been replaced with a hybrid gene of choice.

The invention also provides for the vectors containing the contemplated nucleic acids in a host suitable for expressing the nucleic acids. The vectors containing the nucleic acid segments of interest can be transferred into host cells by well-known methods, which vary depending on the type of cellular host. For example, calcium chloride transformation, transduction, and electroporation are commonly utilized for prokaryotic cells, whereas calcium phosphate, DEAE dextran, or lipofection mediated transfection or electroporation may be used for other cellular hosts.

Alternatively, the nucleic acids of the present invention can be operatively linked to one or more of the functional elements that direct and regulate transcription of the inserted nucleic acid and the nucleic acid can be expressed. For example, a nucleic acid can be operatively linked to a bacterial or phage promoter and used to direct the transcription of the nucleic acid in vitro. A further example includes using a nucleic acid provided herein in a coupled transcription-translation system where the nucleic acid directs transcription and the RNA thereby produced is used as a template for translation to produce a polypeptide. One skilled in the art will appreciate that the products of these reactions can be used in many applications such as using labeled RNAs as probes and using polypeptides to generate antibodies or in a procedure where the polypeptides are being administered to a cell or a subject.

Expression of the nucleic acid, in combination with a vector, can be by either in vivo or in vitro. In vivo synthesis comprises transforming prokaryotic or eukaryotic cells that can serve as host cells for the vector. Alternatively, expression of the nucleic acid can occur in an in vitro expression system. For example, in vitro transcription systems are commercially available which are routinely used to synthesize relatively large amounts of mRNA. In such in vitro transcription systems, the nucleic acid encoding a Cyp polypeptide would be cloned into an expression vector adjacent to a transcription promoter. For example, the Bluescript II cloning and expression vectors contain multiple cloning sites which are flanked by strong prokaryotic transcription promoters. (Stratagene Cloning Systems, La Jolla, Calif.). Kits are available which contain all the necessary reagents for in vitro synthesis of an RNA from a DNA template such as the Bluescript vectors. (Stratagene Cloning Systems, La Jolla, Calif.). RNA produced in vitro by a system such as this can then be translated in vitro to produce the desired Cyp polypeptide. (Stratagene Cloning Systems, La Jolla, Calif.).

Also provided is an isolated Cyp 4A14 polypeptide having the amino acid sequence of SEQ ID NO: 2. Further provided is an isolated Cyp 4A12 polypeptide having the amino acid sequence of SEQ ID NO: 3. The present invention also provides an isolated Cyp 4A11 polypeptide having the amino acid sequence of SEQ ID NO: 5. Also provided is an isolated Cyp 4A22 polypeptide having the amino acid sequence of SEQ ID NO: 7.

As used herein an “isolated polypeptide” means a sequence which is substantially free from the naturally occurring materials with which the amino acid sequence is normally associated in nature. The polypeptides of this invention can comprise the entire amino acid sequence of a Cyp polypeptide or fragments thereof. The polypeptides or fragments thereof of the present invention can be obtained by isolation and purification of the polypeptides from cells where they are produced naturally or by expression of exogenous nucleic acid encoding a Cyp polypeptide. Fragments of a Cyp polypeptide can be obtained by chemical synthesis of peptides, by proteolytic cleavage of the Cyp polypeptide or by synthesis from nucleic acid encoding the portion of interest. The Cyp polypeptide may include conservative substitutions where a naturally occurring amino acid is replaced by one having similar properties. Such conservative substitutions do not alter the function of the polypeptide.

Thus, it is understood that, where desired, modifications and changes may be made in the nucleic acid encoding the polypeptides of this invention and/or amino acid sequence of the polypeptides of the present invention and still obtain a polypeptide having like or otherwise desirable characteristics. Such changes may occur in natural isolates or may be synthetically introduced using site-specific mutagenesis, the procedures for which, such as mis-match polymerase chain reaction (PCR), are well known in the art.

For example, certain amino acids may be substituted for other amino acids in a polypeptide without appreciable loss of functional activity. It is thus contemplated that various changes may be made in the amino acid sequence of a Cyp polypeptide (or underlying nucleic acid sequence) without appreciable loss of biological utility or activity and possibly with an increase in such utility or activity.

These polypeptides can also be obtained in any of a number of procedures well known in the art. One method of producing a polypeptide is to link two peptides or polypeptides together by protein chemistry techniques. For example, peptides or polypeptides can be chemically synthesized using currently available laboratory equipment using either Fmoc (9-fluorenylmethyloxycarbonyl) or Boc (tert -butyloxycarbonoyl) chemistry. (Applied Biosystems, Inc., Foster City, Calif.). One skilled in the art can readily appreciate that a peptide or polypeptide corresponding to a particular protein can be synthesized by standard chemical reactions. For example, a peptide or polypeptide can be synthesized and not cleaved from its synthesis resin whereas the other fragment of a hybrid peptide can be synthesized and subsequently cleaved from the resin, thereby exposing a terminal group which is functionally blocked on the other fragment. By peptide condensation reactions, these two fragments can be covalently joined via a peptide bond at their carboxyl and amino termini, respectively, to form a larger polypeptide. (Grant, ASynthetic Peptides: A User Guide, W. H. Freeman and Co., N.Y. (1992) and Bodansky and Trost, Ed., Principles of Peptide Synthesis, Springer-Verlag Inc., N.Y. (1993)). Alternatively, the peptide or polypeptide can be independently synthesized in vivo as described above. Once isolated, these independent peptides or polypeptides may be linked to form a larger protein via similar peptide condensation reactions.

For example, enzymatic ligation of cloned or synthetic peptide segments can allow relatively short peptide fragments to be joined to produce larger peptide fragments, polypeptides or whole protein domains (Abrahmsen et al. Biochemistry, 30:4151 (1991)). Alternatively, native chemical ligation of synthetic peptides can be utilized to synthetically construct large peptides or polypeptides from shorter peptide fragments. This method consists of a two step chemical reaction (Dawson et al. A Synthesis of Proteins by Native Chemical Ligation, Science, 266:776-779 (1994)). The first step is the chemoselective reaction of an unprotected synthetic peptide-%-thioester with another unprotected peptide segment containing an amino-terninal Cys residue to give a thioester-linked intermediate as the initial covalent product. Without a change in the reaction conditions, this intermediate undergoes spontaneous, rapid intramolecular reaction to form a native peptide bond at the ligation site. Application of this native chemical ligation method to the total synthesis of a protein molecule is illustrated by the preparation of human interleukin 8 (IL-8) (Clark-Lewis et al. FEBS Lett., 307:97 (1987), Clark-Lewis et al., J.Biol.Chem., 269:16075 (1994), Clark-Lewis et al. Biochemistry, 30:3128 (1991), and Rajarathnam et al. Biochemistry, 29:1689 (1994)).

Alternatively, unprotected peptide segments can be chemically linked where the bond formed between the peptide segments as a result of the chemical ligation is an unnatural (non-peptide) bond (Schnolzer et al. Science, 256:221 (1992)). This technique has been used to synthesize analogs of protein domains as well as large amounts of relatively pure proteins with full biological activity (deLisle Milton et al. ATechniques in Protein Chemistry IV, Academic Press, New York, pp. 257-267 (1992)).

The polypeptides of this invention can be linked to another moiety such as a nucleic acid, a protein, a peptide, a ligand, a carbohydrate moiety, viral proteins, a monoclonal antibody, a polyclonal antibody or a liposome.

Also provided by the present invention are antibodies that specifically bind to a Cyp polypeptide of this invention. For example, the antibodies of the present invention can be antibodies that specifically bind to Cyp 4A14, antibodies that specifically bind to Cyp 4A12, antibodies that specifically bind to Cyp 4A11 or antibodies that specifically bind to Cyp 4A22. The antibody (either polyclonal or monoclonal) can be raised to any of the polypeptides provided and contemplated herein, both naturally occurring and recombinant polypeptides, and immunogenic fragments, thereof. The antibody can be used in techniques or procedures such as diagnostics, treatment, or vaccination. Anti-idiotypic antibodies and affinity matured antibodies are also considered.

Antibodies can be made by many well-known methods (See, e.g. Harlow and Lane, “Antibodies; A Laboratory Manual” Cold Spring Harbor Laboratory, Cold Spring Harbor, N.Y., (1988)). Briefly, purified antigen can be injected into an animal in an amount and in intervals sufficient to elicit an immune response. Antibodies can either be purified directly, or spleen cells can be obtained from the animal. The cells can then fused with an immortal cell line and screened for antibody secretion. The antibodies can be used to screen nucleic acid clone libraries for cells secreting the antigen. Those positive clones can then be sequenced. (See, for example, Kelly et al. Bio/Technology, 10:163-167 (1992); Bebbington et al. Bio/Technology, 10:169-175 (1992)). Humanized and chimeric antibodies are also comtemplated in this invention. Heterologous antibodies can be made by well known methods (See, for example, U.S. Pat. Nos. 5,545,806, 5,569,825, 5,625,126, 5,633,425, 5,661,016, 5,770,429, 5,789,650, and 5,814,318)

The phrase “specifically binds” with the polypeptide refers to a binding reaction which is determinative of the presence of the protein in a heterogeneous population of proteins and other biologics. Thus, under designated immunoassay conditions, the specified antibodies bound to a particular protein do not bind in a significant amount to other proteins present in the sample. Selective binding to an antibody under such conditions may require an antibody that is selected for its specificity for a particular protein. A variety of immunoassay formats may be used to select antibodies that selectively bind with a particular protein. For example, solid-phase ELISA immunoassays are routinely used to select antibodies selectively immunoreactive with a protein. See Harlow and Lane “Antibodies, A Laboratory Manual” Cold Spring Harbor Publications, New York, (1988), for a description of immunoassay formats and conditions that could be used to determine selective binding.

The invention also provides a method of identifying an agent capable of enhancing the activity of Cyp 4A14, comprising contacting Cyp 4A14 with a test agent, and determining if the activity of Cyp 4A14 is enhanced as compared to the activity of uncontacted Cyp 4A14, whereby an increase in Cyp4A14 activity indicates that the test agent is capable of enhancing the activity of Cyp 4A14. Such a method may comprise contacting a first Cyp 4A14 sample with a test agent, and determining if the activity of Cyp 4A14 in the first Cyp 4A14 sample is enhanced as compared to the activity of Cyp 4A14 in a second Cyp 4A14 sample which has not been contacted with the test agent, whereby an increase in Cyp4A14 activity in the first sample as compared to the second sample indicates that the test agent is capable of enhancing the activity of Cyp 4A14.

As used herein, the activity of Cyp 4A14, Cyp 4A12, Cyp 4A11 and Cyp 4A22 may be determined in vitro by any assay known to those of skill in the art for Cytochrome p-450 enzymes. One such assay is described in Capdevila et al. (Methods in Enzymology, 187:385-394 (1990)). This reference is incorporated herein, in its entirety. Cyp 4A12 and Cyp 4A11 activity may be assayed specifically by measuring their ability to produce 20-HETE.

The activity of Cyp 4A14, Cyp 4A12, Cyp 4A11 and Cyp 4A22 may also be measured, as used herein, by determining levels of transcription of the relevant gene encoding the Cyp isoform, using, e.g., northern blots. Their activity may also be measured by determining the level of protein expression using, e.g., SDS-PAGE, or antibody-based assays such as the performance of an ELISA using antibodies specific for the relevant Cyp isoform. Such methods are well known to those of ordinary skill in the art.

The invention also provides a method of identifying an agent capable of inhibiting the activity of Cyp 4A14, comprising contacting Cyp 4A14 with a test agent, and determining if the activity of Cyp 4A14 is inhibited as compared to the activity of uncontacted Cyp 4A14, whereby a decrease in Cyp4A14 activity indicates that the test agent is capable of inhibiting the activity of Cyp 4A14. Such a method may, for example, comprise contacting a first Cyp 4A14 sample with a test agent, and determining if the activity of Cyp 4A14 in the first Cyp 4A14 sample is inhibited as compared to the activity of Cyp 4A14 in a second Cyp 4A14 sample which has not been contacted with the test agent, whereby a decrease in Cyp4A14 activity in the first sample as compared to the second sample indicates that the test agent is capable of inhibiting the activity of Cyp 4A14.

The invention also provides a method of identifying an agent capable of enhancing the activity of Cyp 4A12, comprising contacting Cyp 4A12 with a test agent, and determining if the activity of Cyp 4A12 is enhanced as compared the activity of uncontacted Cyp 4A12, whereby an increase in Cyp4A12 activity indicates that the test agent is capable of enhancing the activity of Cyp 4A12. Such a method includes contacting a first Cyp 4A12 sample with a test agent, and determining if the activity of Cyp 4A12 in the first Cyp 4A12 sample is enhanced as compared the activity of Cyp 4A12 in a second Cyp 4A12 sample which has not been contacted with the test agent, whereby an increase in Cyp4A12 activity in the first sample as compared to the second sample indicates that the test agent is capable of enhancing the activity of Cyp 4A12.

The invention also provides a method of identifying an agent capable of inhibiting the activity of Cyp 4A12, comprising contacting Cyp 4A12 with a test agent, and determining if the activity of Cyp 4A12 is inhibited as compared to the activity of uncontacted Cyp 4A12, whereby a decrease in Cyp4A12 activity indicates that the test agent is capable of inhibiting the activity of Cyp 4A12. Such a method includes contacting a first Cyp 4A12 sample with a test agent, and determining if the activity of Cyp 4A12 in the first Cyp 4A12 sample is inhibited as compared to the activity of Cyp 4A12 in a second Cyp 4A12 sample which has not been contacted with the test agent, whereby a decrease in Cyp4A12 activity in the first sample as compared to the second sample indicates that the test agent is capable of inhibiting the activity of Cyp 4A12.

The invention also provides a method of identifying an agent capable of enhancing the activity of Cyp 4A22, comprising contacting Cyp 4A22 with a test agent, and determining if the activity of Cyp 4A22 is enhanced as compared the activity of uncontacted Cyp 4A22, whereby an increase in Cyp4A12 activity indicates that the test agent is capable of enhancing the activity of Cyp 4A22. Such a method includes contacting a first Cyp 4A22 sample with a test agent, and determining if the activity of Cyp 4A22 in the first Cyp 4A22 sample is enhanced as compared the activity of Cyp 4A22 in a second Cyp 4A22 sample which has not been contacted with the test agent, whereby an increase in Cyp4A12 activity in the first sample as compared to the second sample indicates that the test agent is capable of enhancing the activity of Cyp 4A22.

The invention also provides a method of identifying an agent capable of inhibiting the activity of Cyp 4A22, comprising contacting Cyp 4A22 with a test agent, and determining if the activity of Cyp 4A22 is inhibited as compared to the activity of uncontacted Cyp 4A22, whereby a decrease in Cyp4A12 activity indicates that the test agent is capable of inhibiting the activity of Cyp 4A22. Such a method includes contacting a first Cyp 4A22 sample with a test agent, and determining if the activity of Cyp 4A22 in the first Cyp 4A22 sample is inhibited as compared to the activity of Cyp 4A22 in a second Cyp 4A22 sample which has not been contacted with the test agent, whereby a decrease in Cyp4A12 activity in the first sample as compared to the second sample indicates that the test agent is capable of inhibiting the activity of Cyp 4A22.

The invention also provides a method of screening for an agent capable of inhibiting the activating effect of testosterone on the activity of Cyp 4A12, comprising contacting Cyp 4A12 with a test agent in the presence of testosterone, and determining if the activity of Cyp 4A12 is inhibited as compared to the activity of Cyp 4A12 in the presence of testosterone but which has not been contacted with the test agent, whereby a decrease in Cyp 4A12 activity indicates that the test agent is capable of inhibiting the activating effect of testosterone on the activity of Cyp 4A12. Such a method may include contacting a first Cyp 4A12 sample with a test agent in the presence of testosterone, and determining if the activity of Cyp 4A12 is inhibited as compared to (a) the activity of Cyp 4A12 in a second Cyp 4A12 sample, which includes testosterone but which has not been contacted with the test agent, and (b) the activity of Cyp 4A12 in a third Cyp 4A12 sample, which does not include testosterone but which has been contacted with the test agent, whereby a decrease in Cyp4A 12 activity in the first sample as compared to both the second sample and the third sample indicates that the test agent is capable of inhibiting the activating effect of testosterone on the activity of Cyp4A12.

The invention also provides a method of screening for an agent capable of enhancing the activating effect of testosterone on the activity of Cyp 4A12, comprising contacting Cyp 4A12 with a test agent in the presence of testosterone, and determining if the activity of Cyp 4A12 is enhanced as compared to the activity of Cyp 4A12 in the presence of testosterone but which has not been contacted with the test agent, whereby an increase in Cyp4A12 activity indicates that the test agent is capable of enhancing the activating effect of testosterone on the activity of Cyp 4A12. Such a method may include, for example, contacting a first Cyp 4A12 sample with a test agent in the presence of testosterone, and determining if the activity of Cyp 4A12 is enhanced as compared to (a) the activity of Cyp 4A12 in a second Cyp 4A12 sample, which includes testosterone but which has not been contacted with the test agent, and (b) the activity of Cyp 4A12 in a third Cyp 4A12 sample, which does not include testosterone but which has been contacted with the test agent, whereby an increase in Cyp4A12 activity in the first sample as compared to both the second sample and the third sample indicates that the test agent is capable of enhancing the activating effect of testosterone on the activity of Cyp 4A12.

The invention also provides a method of screening for an agent capable of inhibiting the activating effect of testosterone on the activity of human Cyp 4A11, comprising contacting human Cyp 4A11 with a test agent in the presence of testosterone, and determining if the activity of human Cyp 4A11 is inhibited as compared to the activity of human Cyp 4A 11 in the presence of testosterone but which has not been contacted with the test agent, whereby a decrease in human Cyp 4A11 activity indicates that the test agent is capable of inhibiting the activating effect of testosterone on the activity of human Cyp 4A11. Such a method may include, for example, contacting a first human Cyp 4A11 sample with a test agent in the presence of testosterone, and determining if the activity of human Cyp 4A 11 is inhibited as compared to (a) the activity of human Cyp 4A11 in a second human Cyp 4A11 sample, which includes testosterone but which has not been contacted with the test agent, and (b) the activity of human Cyp 4A11 in a third human Cyp 4A11 sample, which does not include testosterone but which has been contacted with the test agent, whereby a decrease in human Cyp 4A11 activity in the first sample as compared to both the second sample and the third sample indicates that the test agent is capable of inhibiting the activating effect of testosterone on the activity of human Cyp 4A11.

The invention also provides a method of screening for an agent capable of enhancing the activating effect of testosterone on the activity of human Cyp 4A11, comprising contacting human Cyp 4A11 with a test agent in the presence of testosterone, and determining if the activity of human Cyp 4A11 is enhanced as compared to the activity of human Cyp 4A11 in the presence of testosterone but which has not been contacted with the test agent, whereby an increase in human Cyp 4A11 activity indicates that the test agent is capable of enhancing the activating effect of testosterone on the activity of human Cyp 4A11. Such a method may include contacting a first human Cyp 4A11 sample with a test agent in the presence of testosterone, and determining if the activity of human Cyp 4A11 is enhanced as compared to (a) the activity of human Cyp 4A11 in a second human Cyp 4A11 sample, which includes testosterone but which has not been contacted with the test agent, and (b) the activity of human Cyp 4A11 in a third human Cyp 4A11 sample, which does not include testosterone but which has been contacted with the test agent, whereby an increase in human Cyp 4A11 activity in the first sample as compared to both the second sample and the third sample indicates that the test agent is capable of enhancing the activating effect of testosterone on the activity of human Cyp 4A11 .

The invention also provides a non-human transgenic mammal comprising a gene encoding murine Cyp 4A14 which has been inactivated or completely deleted. In one example, the non-human transgenic mammal is a mouse. In another example, the endogenous murine Cyp 4A12 gene has also been inactivated or completely deleted, and a copy of the human Cyp 4A11 gene has been introduced into the genome of the mouse and is active in the mouse. In another example, the human Cyp 4A11 gene has been inactivated.

The invention also provides a non-human transgenic mammal comprising a gene encoding murine Cyp 4A12 which has been inactivated. The non-human transgenic mammal can a mouse, rat or a rabbit.

The invention also provides a method of identifying an agent capable of reducing hypertension, comprising administering a test agent to a transgenic mouse comprising a gene encoding murine Cyp 4A14 which has been inactivated or completely deleted, and comparing the blood pressure of the mouse to the blood pressure of the same breed of mouse to which the test agent has not been administered, wherein a lower blood pressure in the first mouse as compared to the second mouse indicates that the test agent is capable of reducing hypertension.

The invention further provides a method of identifying an agent capable of reducing hypertension, comprising administering a test agent to a transgenic mouse comprising a gene encoding murine Cyp 4A14 which has been inactivated or completely deleted, and in which the endogenous murine Cyp 4A12 gene has also been inactivated or completely deleted, and into which a copy of the human Cyp 4A11 gene has been where the Cyp4A11 gene is active in the mouse, and comparing the blood pressure of the mouse to the blood pressure of the same breed of mouse to which the test agent has not been administered, wherein a lower blood pressure in the first mouse as compared to the second mouse indicates that the test agent is capable of reducing hypertension.

A method of identifying a subject having an increased susceptibility for developing hypertension, comprising detecting a mutant Cyp 4A11 polypeptide or a mutated Cyp 4A11 nucleic acid in the subject, thereby identifying a subject having an increased susceptibility for developing hypertension.

The mutant Cyp 4A11 polypeptides that can be detected by the methods of the present invention include, but are not limited to a mutant Cyp 4A 11 polypeptide wherein tryptophan at position 126 of SEQ ID NO: 5 is substituted with arginine (Cyp 4A11/W126→R), a Cyp 4A11 polypeptide, wherein arginine at position 231 of SEQ ID NO: 5 is substituted with cysteine (Cyp 4A1/R231→C), a Cyp 4A11polypeptide, wherein methionine at position 369 of SEQ ID NO: 5 is substituted with arginine (Cyp 4A11/M369→R) and a human Cyp 4A11 polypeptide, wherein leucine at position 509 of SEQ ID NO: 5 is substituted with phenylalanine (Cyp 4A11/L509→F). Mutated Cyp 4A11 nucleic acids encoding Cyp 4A11/W126→R, Cyp 4A11/R231→C, Cyp 4A11/M369→R and Cyp 4A11/L509→F can also be detected by the methods of this invention.

By “increased susceptibility for developing hypertension” is meant a subject who has a greater than normal chance of developing hypertension, compared to the general population. Such subjects include, for example, a subject that harbors a mutation in a Cyp 4A11 gene such that biological activity of Cyp 4A11 is altered.

By “mutated Cyp 4A11 nucleic acid” is meant a nucleic acid having a nucleotide sequence that differs from the sequence of the wild-type Cyp 4A11 nucleic acid. A “mutated nucleic acid” is also a nucleic acid that encodes a Cyp 4A11 polypeptide having an amino acid sequence that differs from the sequence of a wild-type Cyp 4A11 polypeptide. A mutated nucleic acid also includes a nucleic acid having a mutation (relative to the wild-type nucleic acid) in noncoding sequences, such as 5′ or 3′ sequences or intronic sequences. The mutated Cyp 4A11 nucleic acid having a sequence associated with hypertension can comprise a nucleic acid sequence having an insertion mutation, where one or more nucleotides are inserted into the wild-type sequence. The mutated Cyp 4A11 nucleic acid may also comprise a deletion mutation, where one or more nucleotides are deleted from the wild-type sequence. Such a deletion or insertion mutation may, for example, result in a frameshift mutation, altering the reading frame. Frameshift mutations typically result in truncated (that is, prematurely terminated) Cyp 4A11 polypeptide.

The mutated Cyp 4A11 nucleic acid may also comprise a nonsense mutation, that is, a mutation that changes a codon specific for an amino acid to a chain termination codon. Nonsense mutations result in truncated (that is, prematurely terminated) Cyp 4A11 polypeptide. The mutated Cyp 4A11nucleic acid may also comprise a truncation mutation, that is, a mutated Cyp 4A11 nucleic acid which encodes a truncated Cyp 4A11 polypeptide.

A mutation in a Cyp 4A11 nucleic acid can result in a change in a codon such that the mutated codon now encodes a different amino acid. The mutation can result in a polypeptide having a conservative or a non-conservative substitution at the relevant amino acid residue. The mutated Cyp 4A11 nucleic acid and mutant Cyp 4A11 polypeptide that is detected can be from any cause. For example, mutated Cyp 4A11 nucleic acid can be the result of a familial mutation or a sporadic mutation.

The mutated Cyp 4A11 can be detected by utilizing nucleic acid hybridizations techniques. Probes, primers, and oligonucleotides are used for methods involving nucleic acid hybridization, such as: nucleic acid sequencing, reverse transcription and/or nucleic acid amplification by the polymerase chain reaction, single stranded conformational polymorphism (SSCP) analysis, restriction fragment polymorphism (RFLP) analysis, Southern hybridization, Northern hybridization, in situ hybridization, electrophoretic mobility shift assay (EMSA). By “probe,” “primer,” or oligonucleotide is meant a single-stranded DNA or RNA molecule of defined sequence that can base-pair to a second DNA or RNA molecule that contains a complementary sequence (the “target”). The stability of the resulting hybrid depends upon the extent of the base-pairing that occurs. The extent of base-pairing is affected by parameters such as the degree of complementarity between the probe and target molecules and the degree of stringency of the hybridization conditions. The degree of hybridization stringency is affected by parameters such as temperature, salt concentration, and the concentration of organic molecules such as formamide, and is determined by methods known to one skilled in the art. Probes or primers specific for Cyp 4A11 nucleic acids (for example, genes and/or mRNAs) have at least 80%-90% sequence complementarity, preferably at least 91%-95% sequence complementarity, more preferably at least 96%-99% sequence complementarity, and most preferably 100% sequence complementarity to the region of the Cyp 4A11 nucleic acid to which they hybridize. Probes, primers, and oligonucleotides may be detectably-labeled, either radioactively, or non-radioactively, by methods well-known to those skilled in the art.

By “specifically hybridizes” is meant that a probe, primer, or oligonucleotide recognizes and physically interacts (that is, base-pairs) with a substantially complementary nucleic acid (for example, a Cyp 4A11 nucleic acid) under high stringency conditions, and does not substantially base pair with other nucleic acids.

The invention also relates to a method of treating hypertension in a subject comprising inhibiting testosterone activity in the subject.

The invention also relates to a method of treating hypertension in a subject comprising enhancing Cyp 4A14 activity in the subject.

The invention also relates to a method of treating hypertension in a subject comprising inhibiting Cyp4A11 activity in the subject.

The invention also relates to a method of treating hypertension in a subject comprising inhibiting testosterone activity by enhancing 4A14 activity in the subject.

In the present invention, the subject can be any mammal, preferably human, and can include but is not limited to mouse, rat, guinea pig, hamster, rabbit, cat, dog, goat, monkey, horse and chimpanzee.

Optimal dosages used will vary according to the subject being treated and the inhibitor or the enhancing agent being used. The amount of inhibitor or enhancing agent will also vary among individuals on the basis of age, size, weight, condition, etc. One skilled in the art will realize that dosages are best optimized by the practicing physician and methods for determining dose amounts and regimens and preparing dosage forms are described, for example, in Remington's Pharmaceutical Sciences. For example, suitable doses and dosage regimens can be determined by comparison to agents presently used in the treatment or prevention of hypertension.

Typically, the inhibitor or enhancing agent of this invention can be administered orally or parenterally in a dosage range of 0.1 to 100 mg/kg of body weight depending on the clinical response that is to be obtained. Administration of inhibitor or enhancing agent can be stopped completely following a prolonged remission or stabilization of disease signs and symptoms and readministered following a worsening of either the signs or symptoms of the disease, or following a significant change, as determined by routine follow-up hypertension studies well known to a clinician in this field. The inhibitors and enhancers of this invention can also be administered to treat disease states associated with lipid metabolism, pancreatic dysfunction, obesity, type II diabetes and other cardiovascular diseases.

The efficacy of administration of a particular dose of inhibitor or enhancing agent in treating hypertension as described herein can be determined by evaluating the particular aspects of the medical history, the signs, symptoms and objective laboratory tests that have a documented utility in evaluating hypertension. These signs, symptoms and objective laboratory tests will vary as will be well known to any clinician in this field.

Once it is established that disease activity is significantly improved or stabilized by a particular inhibitor or enhancing agent, specific signs, symptoms and laboratory tests can be evaluated in accordance with a reduced or discontinued treatment schedule. If a disease activity recurs, based on standard methods of evaluation of the particular signs, symptoms and objective laboratory tests as described herein, treatment can be reinitiated.

In the present invention, the inhibitors or enhancing agents can be orally or parenterally administered in a carrier pharmaceutically acceptable to human subjects. Suitable carriers for oral or inhaled administration can include one or more of the carriers pharmaceutically acceptable to human subjects. Suitable carriers for oral administration include one or more substances which may also act as a flavoring agents, lubricants, suspending agents, or as protectants. Suitable solid carriers include calcium phosphate, calcium carbonate, magnesium stearate, sugars, starch, gelatin, cellulose, carboxypolymethylene, or cyclodextrans. Suitable liquid carriers may be water, pyrogen free saline, pharmaceutically accepted oils, or a mixture of any of these. The liquid can also contain other suitable pharmaceutical addition such as buffers, preservatives, flavoring agents, viscosity or osmo-regulators, stabilizers or suspending agents. Examples of suitable liquid carriers include water with or without various additives, including carboxypolymethylene as a ph-regulated gel. The inhibitor or enhancing agent may be contained in enteric coated capsules that release the polypeptide into the intestine to avoid gastric breakdown. For parenteral administration, a sterile solution or suspension is prepared in saline that may contain additives, such as ethyl oleate or isopropyl myristate, and can be injected for example, into subcutaneous or intramuscular tissues, as well as intravenously.

By “pharmaceutically acceptable” is meant a material that is not biologically or otherwise undesirable, i.e., the material may be administered to an individual along with the selected compound without causing any undesirable biological effects or interacting in a undesirable manner with any of the other components of the pharmaceutical composition in which it is contained. The carrier may depend on the method of administration and the particular patient. Methods of administration can be oral, sublingual, mucosal, inhaled, absorbed, or by injection. It is also noted that not all methods of administering the inhibitors or enhancing agents described herein require a pharmaceutically acceptable carrier.

The following examples are put forth so as to provide those of ordinary skill in the art with a complete disclosure and description of how the compounds, compositions, articles, devices and/or methods claimed herein are made and evaluated, and are intended to be purely exemplary of the invention and are not intended to limit the scope of what the inventors regard as their invention. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### EXAMPLES

### Cyp 4a14 cDNA Cloning and Expression

The Cyp 4a14 cDNA (2.5 kb), cloned from a mouse liver library (Stratagene), codes for a protein of 507 amino acids with 90% sequence identity to CYP 4A3 and 4A2 (15). A KpnI-XhoI cDNA fragment (1.9 kb) was subcloned into the pBlueBac IV vector (Invitrogen) and expressed by using a commercial sf9/baculovirus expression system (Invitrogen). Recombinant Cyp 4a14 was purified (15) to a specific content of 6 nmol P450/mg of protein and judged to be 70% pure by SDS/PAGE.

### Genomic Cloning and Construction of a Targeting Vector

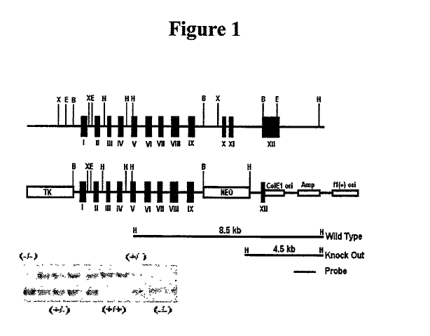

Overlapping genomic clones containing the entire Cyp 4a14 exonic sequences were cloned from a 129/SvJ mouse genomic library (Lambda-FIX II, Stratagene) and partially sequenced. A linearized pNTK targeting vector, in which the sequences coding for the Cyp 4a14 heme-binding peptide (exons 10 and 11) were replaced with a neomycin resistance cassette, resulting in the interruption of in-frame translation at lysine 404 and the insertion of a unique HindIII reporter site for unequivocal genotype analysis (FIG. 1), was electroporated into cultured TL-1129/SvEv Tac mouse embryonic stem cells (ES) and Cyp 4a14 recombinant ES cells identified by Southern blot analysis. A recombinant ES clone carrying a Cyp 4a14 mutant allele was isolated, expanded, and used for blastocyst implantation and the generation of germline chimeras.

### Measurements of Enzyme Activity

Kidney microsomes were isolated from treated and nontreated Cyp 4a14 (+/+) and (−/−) mice (21), suspended (1-2 mg of protein/ml) in 0.05 M Tris·Cl (pH 7.4) containing 0.15 M KCl and 10 mM MgCl2, and incubated with [1¹⁴C]AA or lauric acid (100 μM, 5 μCi/μmol each) and NADPH (1 mM) at 35° C. Reaction products were resolved and quantified as described (21). For antibody inhibition, microsomes were incubated (30 min at 22° C.) with rabbit anti-CYP 4A2 or nonimmune serum (0.1-1 mg of protein/ml) before enzymatic analysis. Recombinant Cyp 4a14 (0.1-1 μM) was incubated with [1-¹⁴C]-labeled AA or lauric acid in the presence of purified P450 reductase, cytochrome b5, dilauroylphosphatidylcholine, and NADPH (1 mM), as described (15, 21). Urine collections (8-12 h) were incubated at 35° C. for 2-3 h with -glucoronidase (22) (Sigma) (1 mg/ml) and, after purification, the levels of 19- and 20-HETE were quantified by mass spectroscopy (22).

### Vascular Physiology Measurements

The arterial blood pressures of conscious 12- to 14-week-old mice were measured by means of a right carotid artery catheter (300-500 μm OD). After surgery (24-48 h), animals were allowed to become familiar with the environment and, after stabilization, their arterial blood pressures were monitored continuously for at least 30 min by using a pressure transducer. Technical limitations impeded the accurate measurement of blood pressures in animals younger than 8 weeks. For measurements of afferent arteriolar diameter, male kidneys were perfused in vitro with a physiological salt solution supplemented with a mixture of L-amino acids (23), and the juxtamedullary vasculature was monitored continuously by videomicroscopy, as described (23). The relationship between afferent arteriolar diameter and perfusion pressure was determined at 80, 120, and 160 mm Hg. Perfusion pressure changes were followed by a 3-min equilibration before steady-state diameter measurements (23).

### Disruption of the Cyp 4a14 Gene Causes Spontaneous Hypertension

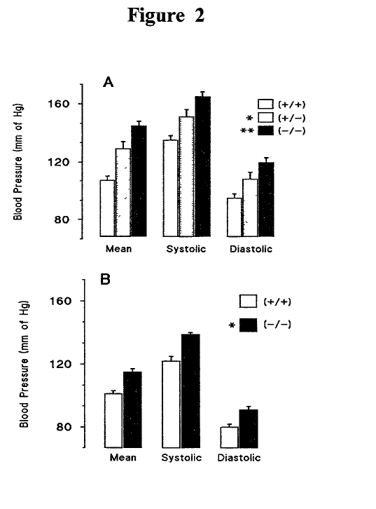

Murine germline chimeras carrying a Cyp 4a14 mutant allele were generated as shown in FIG. 1. By mating to wild-type 129/SvJ mice and genetic selection, isogenic homozygous Cyp 4a14 (+/+) and (−/−) mice [from the progeny of an F2 (+/−)×(+/−) cross] were generated. Initial genotype analysis indicated normal offspring patterns after (+/−)×(+/−) crossings. Male and female 4a14 (−/−) mice developed normally and lacked outward symptoms of disease or organ malformation. Measurements of systemic blood pressure in sexually mature male 4a14 (−/−) (+/−) and (+/+) mice provided decisive evidence of a physiological role for murine 4a P450s in blood pressure control (FIG. 2A). Compared with wild type, Cyp 4a14 (−/−) mice show significant increases in their mean (MABP), systolic, and diastolic arterial blood pressures (FIG. 2A), whereas 4a14 (+/−) animals show intermediate values (FIG. 2A). The 4a14 (−/−) hypertensive phenotype is spontaneous, i.e., does not require experimental manipulations, and is insensitive to dietary salt [i.e., feeding salt diets containing either 3.0 or 0.03% NaCl (wt/wt) for 4-6 weeks had only minor effects on systemic blood pressure]. Furthermore, 4a14 (+/+) and (−/−) mice showed similar plasma levels of Na+, K+, and aldosterone.

### Hypertension in Cyp 4a14 (/) Mice Is Sexually Dimorphic

A large subset of human hypertension is sexually dimorphic, i.e., more severe in males than in females, differences that are minimized after menopause (2, 6-9, 24). Sexual dimorphism is also observed in the hypertensive phenotype of 4a14 (−/−) mice. Blood pressures in female 4a14 (+/+) and (−/−) mice are lower than those of age-matched males, and their pressure differentials are not as pronounced (FIG. 2B). Of interest, disruption of the Cyp 4a14 gene brings the MABPs of knockout females to levels comparable to that of wild-type males [MABPs of 115±2 and 110±4 for Cyp 4a14 (−/−) females and (+/+) males, respectively; n=20; P=0.3) (FIG. 2)]. A similar sexual dimorphism has been observed in SHR rats, an extensively characterized polygenic model of hypertension (24-27).

These gender differences suggested a role for androgens in the Cyp 4a14 (−/−) phenotype and led to analysis of their plasma levels and role in blood pressure regulation. As shown in Table 1, Cyp 4a14 (−/−) males have plasma testosterone (TST) and 5-dihydrotestosterone (DHT) levels twice as high as those of (+/+) mice, demonstrating a role for products of this gene in androgen regulation and the existence of a hitherto unrecognized regulatory loop between the fatty acid hydroxylase and mechanisms that control androgen biosynthesis, metabolism, or degradation. Importantly, neither recombinant Cyp 4a14 nor rat CYPs 4A1 or 4A2 catalyzed TST oxidation, nor was the metabolism of TST by liver microsomes affected by the disruption of the 4a14 gene. Male-specific expression of rat and mouse kidney 4A isoforms and their androgen-dependent regulation have been reported (18, 19).

### Hypertension in Cyp 4a14 (−/−) Mice Is Androgen-Sensitive

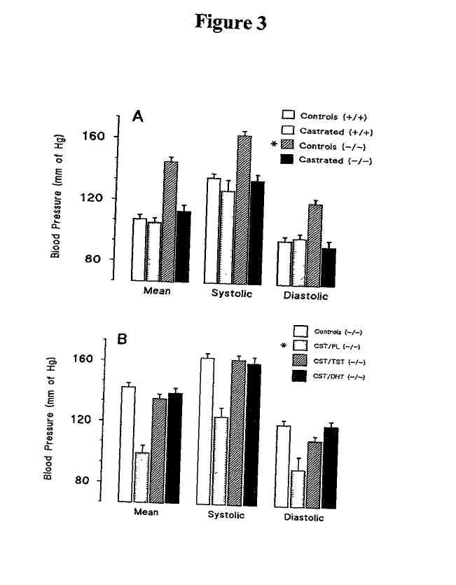

To examine the role of androgens in the Cyp 4a14 (−/−) hypertensive phenotype, 4a14 (+/+) and (−/−) mice were castrated and implanted with either placebo or TST-releasing pellets. Castration markedly reduced the plasma concentrations of DHT and TST in Cyp 4a14 (+/+) and (−/−) mice (Table 1) and normalized the blood pressures of hypertensive 4a14 (−/−) mice (FIG. 3A). On the other hand, castration had a minor effect on the blood pressures of 4a14 (+/+) mice (FIG. 3A), suggesting that plasma androgen levels must reach a threshold before significant changes in blood pressure can be observed. The administration of DHT or TST to castrated 4a14 (−/−) mice raised the plasma levels of these androgens (Table 1) (1.8±0.04 ng TST/ml; n=7) and restored the hypertensive phenotype of castrated 4a14 (−/−) mice (FIG. 3B). These androgen-mediated pressure effects were gender and Cyp 4a14 genotype independent, because the administration of DHT also raised the blood pressures of: (i) control and castrated Cyp 4a14 (+/+) mice (MABPs of 140±5 and 137±4 mm of Hg for control and castrated mice, respectively; n=10) (P 0.001 and 0.0004 for DHT treated vs. nontreated mice, and for castrated and DHT-treated vs. castrated mice, respectively), and (ii) female 4a14 (+/+) or (−/−) mice (MABPs of 133±4 and 132±4 mm of Hg for (−/−) and (+/+) female mice, respectively; n≅9) [P<0.0003 and 0.0006 for DHT-treated (−/−) and (+/+) mice vs. the respective untreated controls]. Hence, the blood pressures of male and female mice are androgen-sensitive, and male Cyp 4a14 (−/−) hypertension is associated with increases in plasma androgens caused by Cyp 4a14 gene-dependent perturbations in the mechanisms that control the circulating levels of these hormones. A similar androgen sensitivity has been reported in SHR rats (25-28). Castration reduces the MABP of male hypertensive SHR rats by 30 to 40 mm of Hg (26-29) and, as with 4a14 (−/−) mice, the normotensive effects of castration are reversed by TST replacement (25-27). Furthermore, androgen administration equalizes the MABP of hypertensive male and female SHR rats (26, 28). The similarities between a component of the hypertensive phenotypes of SHR rats and of P450 4a14 knockout mice support the proposal that P450 4A isoforms contribute to the full development of high blood pressure in adult SHR rats (10).

### The Expression and Activities of the Kidney Cyp 4a AA Monooxygenases Are Androgen-Sensitive

To determine whether the Cyp 4a14 (−/−) hypertension was linked to androgen-mediated changes in renal AA metabolism and 20-HETE formation, microsomal 20-HETE biosynthesis and Cyp 4a expression in the kidneys of control, castrated, and castrated and androgen-treated mice were characterized. Significantly, purified recombinant Cyp 4a14 did not metabolize AA even in the presence of cytochrome b5, excess P450 reductase, GSH, EDTA, and/or sodium cholate (15, 30, 31). The enzyme does, however, catalyze lauric acid oxidation (4.0±0.8 nmol product/min/nmol of P450). Thus, Cyp 4a14 is the closest murine 4a family member to rat CYP 4A2, even though it does not metabolize AA. Compared with normotensive Cyp 4a14 (+/+) controls, kidney microsomes from sexually mature hypertensive 4a14 (−/−) male mice metabolize AA to 20-HETE at significantly higher rates (Table 2). In contrast, 4a14 (+/+) and (−/−) females show nearly undetectable renal AA monooxygenase activities (Table 2). Despite these enzymatic differences, mass spectroscopic quantification of urinary 20-HETE (22) showed its concentrations to be low and similar for the 4a14 (+/+) and (−/−) genotypes (0.18±0.04 and 0.24±0.01 ng/ml of urine for wild-type and knockout mice), indicating that, as with most P450 eicosanoids, the urinary levels of 20-HETE may be controlled by degradation and/or metabolism, as opposed to biosynthetic capacity (10-12, 22).

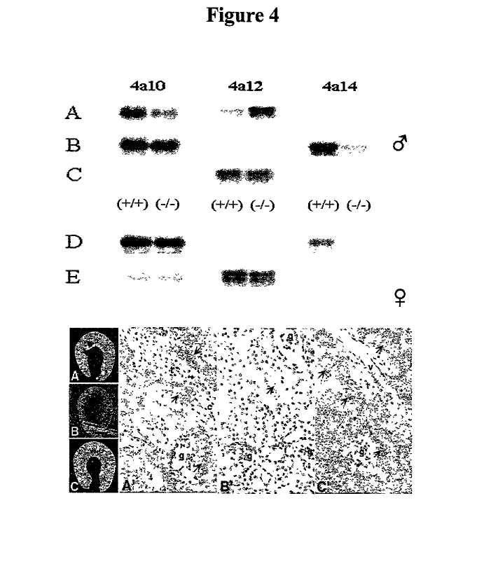

Northern analysis of kidney Cyp 4a isoform expression showed that: (i) Cyp 4a10 is the predominant 4a isoform expressed in the kidneys of wild-type adult males, followed by Cyp 4a12 and low levels of Cyp 4a14 transcripts (FIG. 4). In males, the expression of kidney Cyp 4a14 is variable, age-dependent, and minimized on reaching sexual maturity (not shown). (ii) The female kidney expresses Cyp 4a10 and 4a14 and, as reported (19), lacks detectable Cyp 4a12 transcripts (FIG. 4). (iii) Disruption of the 4a14 gene had little effect on Cyp 4a10 or 4a12 expression by the female kidney (FIG. 4) but causes male-specific up-regulation of the Cyp 4a12 gene and down-regulation of the Cyp 4a10 gene (FIG. 4). Castration drastically decreased renal AA metabolism (Table 2), reduced kidney Cyp 4a12 expression to undetectable levels (FIG. 4), and up-regulated Cyp 4a10 and 4a14 expression (FIG. 4). On the basis of the relative levels of Cyp 4a transcripts and AA monooxygenase activity, kidneys from castrated normotensive 4a14 (+/+) and (−/−) males are similar to those of their corresponding female counterparts (Table 2 and FIG. 4).

Androgen administration to castrated male or female mice minimized Cyp 4a10 and 4a14 expression (FIG. 4) and increased, in a Cyp 4a14 genotype-independent fashion, the kidney expression of Cyp 4a12 and the metabolism of AA to 20-HETE (FIG. 4 and Table 2), indicating that Cyp 4a12 is the isoform responsible for 20-HETE formation. Consistent with this interpretation, an antibody raised against the rat homologue of Cyp 4a14 (CYP 4A2) blocked >90% of the kidney microsomal AA co-hydroxylase of DHT-treated male or female mice. The metabolic and regulatory changes shown in Table 2 and FIG. 4 document an androgen-dependent regulation of renal prohypertensive 20-HETE biosynthesis (10, 11). Data in Tables 1 and 2 and FIG. 4 clearly indicate that, whereas androgen administration induces Cyp 4a12-associated hypertension in females, the modest hypertension in the female 4a14 (−/−) mouse is androgen- and Cyp 4a12-independent. The molecular basis of Cyp 4a14 (−/−) female mice hypertension is unknown but presumably results from factors similar to those responsible for lower blood pressures in hypertensive premenopausal women (8, 9, 24).

Synthetic 20-HETE is a powerful renal vasoconstrictor (10, 11, 23, 32), and its documented role in the regulation of glomerular afferent arteriole tone serves as the basis for its proposed prohypertensive roles (10, 11, 23, 32). In situ hybridization of kidney sections from Cyp 4a14 (−/−) mice by using Cyp 4a12 riboprobes demonstrated that hypertensive control and DHT-treated castrated 4a14 (−/−) mice show abundant and selective expression of 4a 12 transcripts in the renal cortex (Bottom frame, FIG. 4 A and C). In contrast, only background levels of Cyp 4a12 gene expression are seen in castrated normotensive Cyp 4a14 (−/−) animals (Bottom frame, FIGS. 4B and B′, and 5). As shown by increased silver grain density (FIG. 4), expression of the Cyp 4a12 gene in hypertensive control and DHT-treated castrated 4a14 (−/−) mice is mostly restricted to the proximal tubule (Bottom frame, FIG. 4 A′ and C′), whereas thick ascending limbs, collecting ducts, and glomeruli show background expression. The abundance of Cyp 4a12 transcripts in close proximity to the glomeruli microcirculation (Bottom frame, FIG. 4 A and C) shows that 20-HETE biosynthesis is localized in close proximity to the afferent arterioles, a paracrine target for its proposed prohypertensive activity (23, 32, 33).

### Increased Renal Vascular Resistance in Cyp 4a14 (−/−) Mice

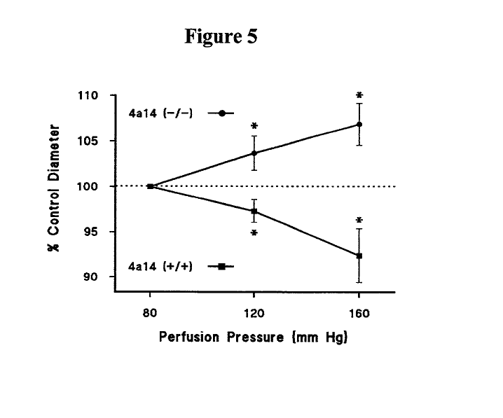

The involvement of an altered renal microvasculature in experimental and human hypertension is well documented (34). Furthermore, elevation in preglomerular vascular resistance may be the determining factor in the decline of renal sodium and water excretion at normotensive pressures and may account for the impaired autoregulatory efficiency frequently observed in chronic hypertension (34). At a perfusion pressure of 80 mm Hg, the kidneys of male Cyp 4a14 (−/−) mice showed a decreased preglomerular vascular diameter [afferent arteriolar diameter: 19±0.5 μm and 17±0.4 μm for Cyp 4a14 (+/+) and 4a14 (−/−) mice, respectively; P 0.05; n≅5 animals, 10 vessels]. This increase in afferent arteriolar resistance may compromise the excretory ability of the Cyp 4a14 (−/−) kidney and may be responsible for the animal's hypertensive phenotype. As reported for the SHR rat (34), Cyp 4a14 (−/−) males show reduced microvascular autoregulatory efficiency. Thus, whereas in Cyp 4a14 (+/+) mice the afferent arteriolar diameter decreased by 8% when renal perfusion pressure was raised from 80 to 160 mm Hg, under identical conditions, it increased by 7% in Cyp 4a14 (−/−) mice (FIG. 5). Similarly, it has been shown that inhibitors of 20-HETE formation also attenuate the pressure response of rat afferent arterioles (23). These results show that increased renal vascular resistance and impaired autoregulatory capacity contribute to the development of hypertension in 4a14 (−/−) mice and that these changes are associated with an increased biosynthesis of vasoconstrictor 20-HETE (10, 11, 34).

In summary, the lack of a functional kidney Cyp 4a14 causes several interrelated metabolic and regulatory effects whose functional manifestations are increased renal vascular resistance, impaired renal hemodynamics, and hypertension. These include increases in: (i) plasma androgens, (ii) Cyp 4a12 gene expression, and (iii) formation of prohypertensive 20-HETE. We postulate that catalytic turnover by Cyp 4a14 generates a yet-to-be-characterized mediator that modulates the levels of circulating androgens. Increased plasma androgen levels induce Cyp 4a12 gene expression and cause attendant increases in proximal tubule 20-HETE biosynthesis, release, and diffusion into the nearby microcirculation. Systemic hypertension results from alterations in nephron hemodynamics, including afferent arteriole autoregulation and renal blood flow (10, 11, 23, 32), caused by increased levels of 20-HETE (10, 11, 32). This interpretation is in agreement with the known renal effects of this eicosanoid (10-12) and provides a molecular/enzymatic description of the relationships between Cyp 4a14 gene function, the regulation of circulating androgens, and renal AA metabolism.

This work establishes that disruption of the murine P450 4a14 gene causes sexually dimorphic hypertension which is, like most human hypertension, more severe in males. P450 4a14 (−/−) mice show increases in plasma testosterone, kidney P450 4a12 expression, and renal arachidonic acid ω-hydroxylase activity. Castration markedly reduces P450 4a12 expression, 20-hydroxy-arachidonic acid formation, and normalizes the blood pressure of hypertensive P450 4a14 (−/−) mice. Androgen replacement restores the hypertensive phenotype, kidney P450 4a12 expression, and arachidonic acid ω-hydroxylation. Similarly, male Sprague-Dawley rats administered testosterone or 5α-dehydrotestoterone became hypertensive (increases of 35-40 mm of Hg in mean arterial blood pressures). This androgen-dependent hypertension is associated with the up-regulation of kidney Cyp 4A8 (the rat homologue of mouse Cyp 4A12) and increased formation of 20-hydroxy-arachidonic acid in the renal micro-circulation.

Human Cyp 4A11 is expressed in the human kidney and catalyzes the formation of pro-hypertensive 20-hydroxyarachidonic acid. Another human 4A isoform, human Cyp 4A22 is inactive towards arachidonic acid.

Throughout this application, various publications are referenced. The disclosures of these publications in their entireties are hereby incorporated by reference into this application in order to more fully describe the state of the art to which this invention pertains.

It will be apparent to those skilled in the art that various modifications and variations can be made in the present invention without departing from the scope or spirit of the invention. Other aspects of the invention will be apparent to those skilled in the art from consideration of the specification and practice of the invention disclosed herein. It is intended that the specification and examples be considered as exemplary only, with a true scope and spirit of the invention being indicated by the claims which follow.

### REFERENCES

1. Lifton, R. P. (1996) Science 272, 676-680.

2. Garbers, D. L. & Dubois, S. K. (1999) Annu. Rev. Biochem. 68, 127-155.

3. Dominiczak, A. F. , Negrin, D. C., Clark, J. S., Brosnan, J. M. , McBride, M. W. & Alexander, Y. (2000) Hypertension 35, 164-172.

4. Pratt, R. E. & Dzau, V. J. (1999) Hypertension 33, 238-247.

5. Halushka, M. K., Fan, J. -B., Bentley, K., Hsie, L. , Shen, N., Weder, A., Cooper, R. , Lipshutz, R. & Chakravarti, A. (1999) Nat. Genet. 22, 239-247.

6. Reckelhoff, J. F. & Granger, J. P. (1999) Clin. Exp. Pharmacol. Physiol. 26, 127-131.

7. August, P. (1999) J. Clin. Endocrinol. Metab. 84, 3451-3452.

8. Chen, Y. F. (1996) Curr. Opin. Nephrol. Hypertens. 5, 181-185.

9. Mantzoros, C. S., Georgiadis, E. I., Young, R. , Evagelopoulou, C., Khoury, S., Hatsilambros, N. & Sowers, J. R. (1995) Am. J. Hypertens. 8, 606-614.

10. McGiff, J. C. & Quilley, J. (1999) Am. J. Physiol. 277, R607-R623.

11. Harder, D. R., Lange, A. R., Gebremedhin, D., Birks, E. K. & Roman, R. J. (1997) J. Vasc. Res. 34, 237-243.

12. Capdevila, J. H., Falck, J. R. & Harris, R. C. (2000) J. Lipid Res. 41, 163-181.

13. Su, P., Kaushal, K. M. & Kroetz, D. L. (1998) Am. J. Physiol. 275, R426-R438.

14. Wang, M. H., Guan, H., Nguyen, X., Zand, B. A., Nasjletti, A. & Laniado-Schwartzman, M. (1999) Am. J. Physiol 276, F246-F253.

15. Helvig, C., Dishman, E. & Capdevila, J. H. (1998) Biochemistry 37, 12546-12558.

16. Johnson, E. F., Palmer, C. N., Griffin, K. J. & Hsu, M. H. (1996) FASEB J. 10, 1241-1248.

17. Honkakoski, P. & Negishi, M. (2000) Biochem. J. 347, 321-337.

18. Sundseth, S. S. & Waxman, D. J. (1992) J. Biol. Chem. 267, 3915-3921.

19. Heng, Y. M. , Kuo, S., Jones, P. S., Savory, R. , Schultz, R. M. , Tomlinson, S. R., Gray, J. B. & Bell, D. R. (1997) Biochem. J. 325, 741-749.

20. Kroetz, D. L. , Yook, P., Costet, P., Bianchi, P. & Pineau, T. (1998) J. Biol. Chem. 20, 31581-31589.

21. Capdevila, J. H., Falck, J. R., Dishman, E. & Karara, A. (1990) Methods Enzymol. 187, 385-394.

22. Prakash, C., Zhang, J. Y. , Falck, J. R. , Chaunhan, K. & Blair, I. A. (1992) Biochem. Biophys. Res. Commun. 185, 728-733.

23. Imig, J. D., Falck, J. R. & Inscho, E. W. (1999) Br. J. Pharmacol. 127, 1399-1405.

24. Phillips, G. B., Jing, T. Y. & Laragh, J. H. (1997) J. Hum. Hypertens. 11, 523-526.

25. Chen, Y. F. & Meng, Q. C. (1991) Life Sci. 48, 85-96.

26. Turner, M. E. , Johnson, M. L. & Ely, D. L. (1991) Hypertension 17, 1097-110.

27. Reckelhoff, J. F., Zhang, H., Srivastava, K. & Granger, J. P. (1999) Hypertension 34, 920-923.

28. Masabuchi, Y. , Kumai, T. , Uematsu, A., Komoriyama, K. & Hirai, M. (1982) Acta Endocrinol. 101, 154-160.

29. Ganten, U., Schroder, G. , Witt, M. , Zimmermann, F., Ganten, D. & Stock, G. (1989) J. Hypertens. 7, 721-726.

30. Hoch, U., Zhang, Z. , Kroetz, D. L. & Ortiz de Montellano, P. R. (2000) Arch. Biochem. Biophys. 373, 63-71.

31. Lasker, J. M. , Chen, B. W., Wolf, I., Bloswick, B. P., Wilson, P. D. & Powel, P. K. (2000) J. Biol. Chem. 275, 4118-4126.

32. Imig, J. D. , Zou, A. P., Stec, D. E. , Harder, D. R. , Falck, J. R. & Roman, R. J. (1996) Am. J. Physiol. 270, R217-R227.

33. Navar, L. G. , Inscho, E. W. , Majid, D. S. A., Imig, J. D. , Harrison-Bernard, L. M. & Mitchell, K. D. (1996) Physiol. Rev. 76, 425-536.

34. Cowley, A. W. & Roman, R. J. (1996) J. Am. Med. Assoc. 275, 1581-1589.

35. Imaoka, S., Ogawa, H., Kimura, S. & Gonzalez, F. J. (1993) DNA Cell Biol. 12, 893-899.

TABLE 1
Plasma androgen levels in Cyp 4a14 (+/+) and (−/−) male mice

| | | |
|---|---|---|
| Source of plasma | DHT | TST |

| Control | (+/+) | 0.44 ± 0.05 | 1.16 ± 0.1 |
|---|---|---|---|
| | (−/−) | 1.02 ± 0.1* | 2.06 ± 0.3* |
| CST/PL | (+/+) | ≦0.05^{†} | ≦0.20^{†} |
| | (−/−) | ≦0.05^{†} | ≦0.20^{†} |
| CST/DHT | (+/+) | 2.64 ± 0.3** | 0.89 ± 0.1 |
| | (−/−) | 2.01 ± 0.1** | 0.82 ± 0.1 |
| Fourteen-week-old male Cyp 4a14 (+/+) and (−/−) mice were castrated and, 10 days later, implanted with either placebo (CST/PL) or DHT (CST/DHT)-releasing pellets (21-day pellets, 5 mg DHT/day; Innovative Research of America, Sarasota, FL). Ten days after implantation, plasma samples were analyzed for TST and DHT levels by RIA using commercially available kits. Values (in nanograms/milliliters of plasma) are the | | | |
| # mean ± SE of at least 31 different animals for control Cyp 4a14 (+/+) and (−/−) mice and of at least 10 different animals for the rest. Significantly different from control wild type: | | | |

*P ≦ 0.0009 and P ≦ 0.005 for DHT and TST, respectively. Significantly different from control wild-type and knockout mice, respectively:
**P ≦ 2 × 10⁻⁴ and P ≦ 1 × 10⁻³ for castrated DHT-treated (+/+) and (−/−) mice.
^{†}, below assay detection limit.

TABLE 2
The microsomal Arachidonic acid ω-hydroxylase of
mouse kidney microsomes

| Microsomes | | ω-Hydroxylase rate | (20-HETE) % of total |
|---|---|---|---|
| Males | | | |
| Control | (+/+) | 38 ± 2 | 86 |
| Control | (−/−) | 85 ± 9* | 84 |
| CST/PL^{†} | (+/+) | <0.2 | — |
| CST/PL^{†} | (−/−) | <0.2 | — |
| CST/DHT | (+/+) | 228 ± 35** | 82 |
| CST/DHT | (−/−) | 225 ± 23 | 82 |
| Females | | | |
| Placebo | (+/+) | <0.2 | — |
| Placebo^{†} | (−/−) | <0.2 | — |
| DHT | (+/+) | 140 ± 3 | 76 |
| DHT | (−/−) | 164 ± 3 | 77 |
| Fourteen-week-old castrated male and female Cyp 4a14 (+/+) and (−/−) mice were implanted with either placebo (CST/PL) or DHT-releasing pellets (CST/DHT) (Table 1). Ten days later, kidney microsomes were incubated with AA, as described in Methods. Rates, in picomoles of product formed per minute per milligram of microsomal protein, were calculated from the corresponding time courses | | | |
| # of product formation. Values are averages ± SE calculated from at least six (males) or three (females) different experiments. Significantly different from control wild type: | | | |

*, P ≦ 1 × 10⁻⁴ and P ≦ 5 × 10⁻⁴ for total AA metabolism and 20-HETE formation, respectively. Significantly different from control wild-type and knockout mice, respectively:
**, P ≦ 7 × 10⁻⁴ and P ≦ 9 × 10⁻⁴ for castrated DHT-treated (+/+) and (−/−) mice.
†Total reaction rates below detection limits.

What is claimed is:

1. A method of enhancing the activity of Cyp 4A14 by administering an agent that enhances the activity of Cyp 4A14.

2. A method of inhibiting the activity of Cyp 4A14 by administering an agent that inhibits the activity of Cyp 4A14.

3. A method of inhibiting the activity of testosterone by administering an agent that enhances the activity of Cyp 4A14.

4. A method of enhancing the activity of testosterone by administering an agent that inhibits the activity of Cyp 4A14.

5. A method of enhancing the activity of Cyp 4A12 by administering an agent that enhances the activity of Cyp 4A12.

6. A method of inhibiting the activity of Cyp 4A12 by administering an agent that inhibits the activity of Cyp 4A12.

7. A method of inhibiting the activity of Cyp 4A12 by administering an agent that inhibits the activity of testosterone.

8. A method of enhancing the activity of Cyp 4A12 by administering an agent that enhances the activity of testosterone.

9. A method of enhancing the activity of Cyp 4A12 by administering an agent that inhibits the activity of Cyp 4A14.

10. A method of inhibiting the activity of Cyp 4A12 by administering an agent that enhances the activity of Cyp 4A14.

11. A method of enhancing the activity of human Cyp 4A11 by administering an agent that enhances the activity of human Cyp 4A11.

12. A method of inhibiting the activity of human Cyp 4A11 by administering an agent that inhibits the activity of human Cyp 4A11.

13. A method of enhancing the activity of human Cyp 4A11 by administering an agent that inhibits the activity of Cyp 4A14.

14. A method of inhibiting the activity of human Cyp 4A11 by administering an agent that enhances the activity of Cyp 4A14.

15. A method of inhibiting the activity of human Cyp 4A11 by administering an agent that inhibits the activity of testosterone.

16. A method of enhancing the activity of human Cyp 4A11 by administering an agent that enhances the activity of testosterone.

17. A method of enhancing the activity of human Cyp 4A11 by administering an agent that inhibits the activity of Cyp 4A14.

18. A method of inhibiting the activity of human Cyp 4A11 by administering an agent that enhances the activity of Cyp 4A14.

19. A method of identifying an agent capable of enhancing the activity of Cyp 4A14, comprising contacting Cyp 4A14 with a test agent, and determining if the activity of Cyp 4A14 is enhanced as compared to the activity of uncontacted Cyp 4A14, whereby an increase in Cyp4A14 activity indicates that the test agent is capable of enhancing the activity of Cyp 4A14.

20. A method of identifying an agent capable of inhibiting the activity of Cyp 4A14, comprising contacting Cyp 4A14 with a test agent, and determining if the activity of Cyp 4A14 is inhibited as compared to the activity of uncontacted Cyp 4A14, whereby a decrease in Cyp4A14 activity indicates that the test agent is capable of inhibiting the activity of Cyp 4A14.

21. A method of identifying an agent capable of enhancing the activity of Cyp 4A 12, comprising contacting Cyp 4A 12 with a test agent, and determining if the activity of Cyp 4A12 is enhanced as compared to the activity of uncontacted Cyp 4A12, whereby an increase in Cyp4A12 activity indicates that the test agent is capable of enhancing the activity of Cyp 4A12.

22. A method of identifying an agent capable of inhibiting the activity of Cyp 4A 12, comprising contacting Cyp 4A 12 with a test agent, and determining if the activity of Cyp 4A12 is inhibited as compared to the activity of uncontacted Cyp 4A12, whereby a decrease in Cyp4A12 activity indicates that the test agent is capable of inhibiting the activity of Cyp 4A12.

23. A method of screening for an agent capable of inhibiting the activating effect of testosterone on the activity of Cyp 4A12, comprising contacting Cyp 4A12 with a test agent in the presence of testosterone, and determining if the activity of Cyp 4A12 is inhibited as compared to the activity of Cyp 4A12 in the presence of testosterone but which has not been contacted with the test agent, whereby a decrease in Cyp 4A12 activity indicates that the test agent is capable of inhibiting the activating effect of testosterone on the activity of Cyp 4A12.

24. A method of screening for an agent capable of enhancing the activating effect of testosterone on the activity of Cyp 4A12, comprising contacting Cyp 4A12 with a test agent in the presence of testosterone, and determining if the activity of Cyp 4A12 is enhanced as compared to the activity of Cyp 4A12 in the presence of testosterone but which has not been contacted with the test agent, whereby an increase in Cyp4A12 activity indicates that the test agent is capable of enhancing the activating effect of testosterone on the activity of Cyp 4A12.

25. A method of screening for an agent capable of inhibiting the activating effect of testosterone on the activity of human Cyp 4A11, comprising contacting human Cyp 4A11 with a test agent in the presence of testosterone, and determining if the activity of human Cyp 4A11 is inhibited as compared to the activity of human Cyp 4A11 in the presence of testosterone but which has not been contacted with the test agent, whereby a decrease in human Cyp 4A11 activity indicates that the test agent is capable of inhibiting the activating effect of testosterone on the activity of human Cyp 4A11.

26. A method of screening for an agent capable of enhancing the activating effect of testosterone on the activity of human Cyp 4A11, comprising contacting human Cyp 4A11 with a test agent in the presence of testosterone, and determining if the activity of human Cyp 4A11 is enhanced as compared to the activity of human Cyp 4A11 in the presence of testosterone but which has not been contacted with the test agent, whereby an increase in human Cyp 4A11 activity indicates that the test agent is capable of enhancing the activating effect of testosterone on the activity of human Cyp 4A11.

27. A non-human transgenic mammal comprising a gene encoding murine Cyp 4A14 which has been inactivated or completely deleted.

28. The non-human transgenic mammal of claim 27, wherein the non-human transgenic mammal is a mouse.

29. A non-human transgenic mammal comprising a gene encoding murine Cyp 4A12 which has been inactivated.

30. The non-human transgenic mammal of claim 29, wherein the non-human transgenic mammal is a mouse.

31. The non-human transgenic mouse of claim 28, wherein the endogenous murine Cyp 4A12 gene has also been inactivated or completely deleted, and wherein a copy of the human Cyp 4A11 gene has been introduced into the genome of the mouse and is active in the mouse.

32. The mouse of claim 3 1, wherein the human Cyp 4A11 gene has been inactivated.

33. A method of identifying an agent capable of reducing hypertension, comprising administering a test agent to the mouse of claim 28, and comparing the blood pressure of the mouse to the blood pressure of a mouse of claim 28 to which the test agent has not been administered, wherein a lower blood pressure in the first mouse as compared to the second mouse indicates that the test agent is capable of reducing hypertension.

34. A method of identifying an agent capable of reducing hypertension, comprising administering a test agent to the mouse of claim 31 , and comparing the blood pressure of the mouse to the blood pressure of a mouse of claim 31 to which the test agent has not been administered, wherein a lower blood pressure in the first mouse as compared to the second mouse indicates that the test agent is capable of reducing hypertension.

35. A method of treating hypertension in an individual comprising inhibiting testosterone activity in the individual.

36. A method of treating hypertension in an individual comprising enhancing Cyp 4A14 activity in the individual.

37. A method of treating hypertension in an individual comprising inhibiting Cyp4A11 activity in the individual.

38. A method of treating hypertension in an individual comprising inhibiting testosterone activity by enhancing 4A14 activity in the individual.

39. A method of identifying a subject having an increased susceptibility for developing hypertension, comprising detecting a mutant Cyp 4A11 polypeptide or a mutated Cyp 4A11 nucleic acid in the subject, thereby identifying a subject having an increased susceptibility for developing hypertension.

40. The method of claim 39, wherein the mutated nucleic acid encodes Cyp 4A11/W126→R.

41. The method of claim 39, wherein the mutated nucleic acid encodes Cyp 4A11/R231→C.

42. The method of claim 39, wherein the mutated nucleic acid encodes Cyp 4A11/M369→R.

43. The method of claim 39, wherein the mutated nucleic acid encodes Cyp 4A11/L509→F.
